(19) United States
(12) Patent Application Publication
(10) Pub. No.: US 2014/0134283 A1
(43) Pub. Date: May 15, 2014

(54) Compositions and Methods for Affecting Mental State and Body Composition

(71) Applicant: USPLABS. LLC, Dallas, TX (US)

(72) Inventors: Jacobo Edward Geissler, Dallas, TX (US); Jiang Zhou, Beijing (CN); Yang Jiang, Beijing (CN); Li Zhou, Beijing (CN)

(73) Assignee: USPlabs. LLC, Dallas, TX (US)

(21) Appl. No.: 13/801,217

(22) Filed: Mar. 13, 2013

Related U.S. Application Data
(60) Provisional application No. 61/726,784, filed on Nov. 15, 2012.

(51) Int. Cl. A61K 31/165; A61K 31/522; A61K 31/7068; A23L 1/30; A61K 36/88; A61K 36/74; A61K 31/472; C07C 231/14; A61K 36/48

(52) U.S. Cl. CPC A61K 31/165; C07C 231/14; A61K 31/522; A61K 31/7068; A61K 36/48; A61K 36/88; A61K 36/74; A61K 31/472; A23L 1/3002
USPC 424/757; 514/263.31; 514/49; 514/617; 564/182

(57) ABSTRACT

The invention discloses compositions, including nutritional products and dietary supplements, comprising aegeline, which is a compound that occurs naturally in the Bael plant. The invention also describes methods comprising the administration of aegeline to mammals. The methods result in increased mental stamina, focus, and energy level, improved mood, increased thermogenesis, increased libido, and anabolic effects and increased strength output and/or muscle mass. These effects can improve cognition, influence body composition, promote weight loss, and/or promote fitness and well-being. A novel method for the synthesis of aegeline is also provided.

### CROSS-REFERENCE TO RELATED APPLICATIONS

The present application claims the benefit under 35 U.S.C. §119(e) of U.S. Provisional Application No. 61/726,784, filed Nov. 15, 2012, the entirety of which is incorporated herein by reference.

### BACKGROUND OF THE INVENTION

1. Field of the Invention

The field of the invention relates to nutritional products and dietary supplements for improving a mammal's mental state and body composition.

2. Background

The literature has long documented the health benefits of certain herbs, botanicals, spices, plant extracts, and natural food products. These beneficial health effects are likely due to the variety of active compounds found to naturally occur in these plant materials. Such naturally occurring, active compounds include phthallides, polyacetylenes, phenolic acids, flavonoids, coumarins, capsicinoids, triterpenoids, sterols, monoterpenes, and alkaloids, among others. Caffeine, for example, is a methylxanthine found in the seeds, leaves and fruit of some plants. It is commonly ingested for its stimulant effects. See, e.g., Smith, A., Effects of caffeine on human behavior, Food Chem. Tox. 40: 1243-55 (2002).

While plants have been consumed to achieve beneficial effects, there remains a need to characterize the beneficial effects that could result from isolated, purified, or compounded substances that are found within the plants. And for any particular desired effect or combination of effects, there remains a need to identify and isolate naturally occurring compounds that could be useful to achieve that effect or combination of effects. Moreover, while the activities of certain naturally occurring compounds such as caffeine have been documented, studies have revealed the shortcomings of such compounds. In the case of caffeine, its stimulant effects are only transient and of short duration. Also, at high doses, caffeine can cause nervousness, agitation, tremors, and anxiety; caffeine can similarly cause adverse behavioral effects in individuals who are sensitive to caffeine. See, e.g., Acheson et al., Metabolic effects of caffeine in humans: lipid oxidation or futile cycling?, Am. J. Clin. Nutr. 79: 40-6 (2004); Smith (2002); Kaplan et al., Dose-dependent pharmacokinetic and psychomotor effects of caffeine in humans, J. Clin. Pharmacol. 37: 693-703 (1997), and references cited therein.

The plant Aegle marmelos contains various classes of compounds, including coumarins, alkaloids, tannins, carotenoids, and terpenoids, among others. See Yadav and Chanotia, Phytochemical and pharmacological profile of leaves of Aegle Marmelos Linn, Pharma Rev., November-December, 144-49 (2009); Maity et al., Biological activities of crude extracts and chemical constituents of Bael, Aegle marmelos (L.) Corr., Indian J. Exp. Bio., 47: 849-61 (2009); Sharma et al., A review on Bael tree, Nat. Prod. Radiance, 6: 171-78 (2007). Among the compounds detected in the plant is aegeline. The present invention demonstrates the unique and unexpected activity profiles of aegeline, and further shows that this compound overcomes some of the shortcomings of other naturally occurring compounds such as caffeine. Thus, the present invention provides a naturally occurring compound that exhibits unexpected and improved activity profiles when used in nutritional products and dietary supplements.

### BRIEF SUMMARY OF THE INVENTION

The present invention relates to the unexpected cognitive, thermogenic, and anabolic effects of nutritional products and dietary supplements containing N-[2-hydroxy-2(4-methoxyphenyl) ethyl]-3-phenyl-2-propenamide (e.g., as an isolated, purified, extracted, and/or synthesized compound). This compound is also called aegeline, and it exhibits unique and desirable effects when administered to mammals in sufficient quantity. In particular, administration of aegeline is effective to increase or improve mental stamina, focus, energy level and/or mood; increase thermogenesis; increase libido; and/or improve muscular performance.

Certain aspects of the present invention relate to compositions comprising aegeline. In certain embodiments, the compositions comprise aegeline and one or more other substances selected from the group consisting of caffeine, theophylline, paraxanthine, phenethylamine, arginine α-ketoglutarate, tyrosine, n-acetyl-l-tyrosine, creatine, β-alanine, caffeic acid, citicoline, bauhinia purpurea L. extract, bacopa monnieri extract, schisandra chinensis extract, hemerocallis fulva extract, cirsium oligophyllum extract, higenamine (also known as norcoclaurine), rauwolscine, and yohimbine. In some embodiments, the compositions comprise aegeline and one or more other substances selected from the group consisting of caffeine, bauhinia purpurea L. extract, hemerocallis fulva extract, yohimbe (pausinystalia johimbe) extract, and norcoclaurine hydrochloride. In further embodiments, the invention relates to compositions comprising aegeline, bauhinia purpurea L. extract, hemerocallis fulva extract, yohimbe (pausinystalia johimbe) extract, and higenamine. In additional embodiments, the compositions of the present invention comprise aegeline and caffeine. Accordingly, certain embodiments relate to compositions comprising aegeline, caffeine, bauhinia purpurea L. extract, hemerocallis fulva extract, yohimbe (pausinystalia johimbe) extract, and higenamine or norcoclaurine. In addition, certain embodiments of the present invention relate to compositions comprising aegeline, caffeine, arginine α-ketoglutarate, creatine, β-alanine, and schisandra chinensis extract. In other embodiments, the present invention relates to compositions comprising aegeline, arginine α-ketoglutarate, creatine, β-alanine, and schisandra chinensis extract. In additional embodiments, the invention relates to compositions comprising aegeline and citicoline. Thus embodiments of the present invention also relate compositions comprising aegeline and one or more substances selected from the group consisting of caffeine, citicoline, bauhinia purpurea L. extract, hemerocallis fulva extract, yohimbe extract, and higenamine.

In certain embodiments, the present invention relates to compositions comprising aegeline, wherein the amount of aegeline is an amount ranging from about 10% to about 100% of the composition by weight. In further embodiments, the amount of aegeline is an amount ranging from about 15% to about 100% of the composition by weight, and in other embodiments the amount of aegeline ranges from about 10% to about 50% of the composition by weight. In further embodiments, the amount of aegeline represents about 15% to about 25% of the composition by weight. In still further embodiments, the compositions of the present invention also comprise caffeine. In certain of these embodiments, caffeine is present in an amount ranging from about 30% to about 50% of the composition by weight. The compositions of the present invention may further comprise one or more other substances, and the amount of one or more of these other substances may, in some embodiments, be an amount ranging from about 0.5% to about 60% of the composition by weight.

In certain embodiments, including the embodiments above, the present invention relates to compositions comprising aegeline, wherein the aegeline is present in an amount ranging from about 30 mg to about 3000 mg. In further embodiments, the aegeline is present in an amount ranging from about 40 mg to about 2000 mg. In still further embodiments, the aegeline is present in an amount ranging from about 80 mg to about 1000 mg. Specific embodiments of the present invention relate to compositions comprising aegeline, wherein the aegeline is present in an amount of about 80 mg, about 100 mg, about 120 mg, about 150 mg, about 200 mg, or about 400 mg.

In other embodiments, the present invention relates to compositions comprising aegeline, wherein the aegeline is present in an amount ranging from about 30 mg to about 300 mg. In certain embodiments, the amount of aegeline is an amount of about 40 mg, about 80 mg, or about 120 mg.

In alternative embodiments, the present invention relates to compositions comprising aegeline, wherein the aegeline is present in an amount ranging from about 200 mg to about 3000 mg. In further of these embodiments, the aegeline is present in an amount ranging from about 400 mg to about 3000 mg, and in still further embodiments, the aegeline is present in an amount ranging from about 600 mg to about 800 mg. In other embodiments, the aegeline is present in amount ranging from about 200 mg to about 600 mg.

In aspects relating to compositions comprising aegeline in combination with one or more other substances, in certain embodiments, the compositions comprise aegeline and caffeine, wherein the aegeline is present in an amount ranging from about 40 mg to about 800 mg and the caffeine is present in an amount ranging from about 50 mg to about 500 mg. In other embodiments, the compositions comprise aegeline and one or more other substances, wherein the aegeline is present in an amount ranging from about 40 mg to about 800 mg and the one or more other substances is present in an amount ranging from about 0.5 mg to about 500 mg; in further embodiments the one or more other substances is present in an amount ranging from about 0.5 mg to about 150 mg. In certain of these embodiments, the one or more other substances is selected from the group consisting of citicoline, bauhinia purpurea L. extract, hemerocallis fulva extract, yohimbe (pausinystalia johimbe) extract, and norcoclaurine or higenamine. In further embodiments, the compositions also comprise caffeine. In certain of these embodiments, the caffeine is present in an amount ranging from about 100 mg to about 200 mg.

Certain aspects of the present invention also relate to the various forms of aegeline. Accordingly, in certain embodiments, the compositions of the present invention comprise aegeline, wherein the aegeline is selected from the group consisting of (R)-aegeline; (S)-aegeline; a racemic mixture of (R)-aegeline and (S)-aegeline; a mixture of (R)-aegeline and (S)-aegeline, wherein the mixture is enriched for (R)-aegeline; and a mixture of (R)-aegeline and (S)-aegeline, wherein the mixture is enriched for (S)-aegeline. In specific embodiments, the aegeline is (R)-aegeline. Certain aspects of the present invention also relate to various salts of aegeline. In specific embodiments, the aegeline is aegeline succinate.

In addition, certain aspects of the present invention relate formulations or preparations comprising aegeline. In certain embodiments, the compositions of the present invention are formulated as an oral dosage form. In further embodiments, the oral dosage form is selected from the group consisting of tablet, capsule, gel cap, powder, gum, gel (including, e.g., paste), and solution.

In certain aspects, the present invention relates to dietary supplements and nutritional products comprising aegeline. Accordingly, some embodiments, including embodiments of the combinations described herein, relate to compositions that are dietary supplements. In certain embodiments, the dietary supplements are formulated as oral dosage forms. In further embodiments, an oral dosage form is selected from the group consisting of tablet, capsule, gel cap, powder, gum, gel and solution, and specific embodiments of the invention relate to an oral dosage form that is a capsule. In additional embodiments, the composition further comprises one or more other ingredients, including, for example, one or more excipients.

Other aspects of the present invention relate to methods and uses involving aegeline administration. Certain aspects relate to a method for increasing mental stamina in a mammal comprising administering an effective amount of aegeline. In specific embodiments, the mammal is a human. In additional embodiments, the amount of aegeline is also effective to increase focus of the human. In further embodiments, the amount of aegeline is also effective to increase energy level of the human, and in still further embodiments the amount of aegeline is also effective to improve mood of the human. The amount of aegeline in other embodiments may also be effective to increase thermogenesis in the human, and the amount of aegeline in additional embodiments may also be effective to increase strength output of the human.

Additional aspects of the present invention relate to methods for improving cognition in a human, comprising administering an amount of aegeline, wherein the amount of aegeline results in an effect selected from the group consisting of an increase in mental stamina, an increase in focus, an increase in energy level, and an improvement in mood. Accordingly, some aspects of the invention relate to methods for improving cognition in a human, comprising administering an amount of aegeline effective to increase mental stamina of the human. Other aspects relate to methods for improving cognition in a human, comprising administering an amount of aegeline effective to increase focus of the human. Additional aspects of the invention relate to methods for improving mood of a human, comprising administering an effective amount of aegeline.

Other aspects of the present invention relate to methods for increasing thermogenesis in a mammal. Accordingly, certain embodiments of the present invention relate to methods for increasing thermogenesis in a human, comprising administering an effective amount of aegeline. In additional embodiments, the invention relates to methods for affecting energy balance in a human, comprising administering an amount of aegeline effective to increase thermogenesis.

Other aspects of the present invention relate to methods for increasing libido in a man, comprising administering an effective amount of aegeline. Thus, in certain embodiments of the invention, the mammal is a human male, and the amount of aegeline that is administered is an amount effective to increase libido in the human male.

Additional aspects of the present invention relate to administering an amount of aegeline effective to produce anabolic effects. Accordingly, certain embodiments of the present invention relate to methods comprising administering an amount of aegeline effective to increase strength output in a mammal. In further embodiments, the invention relates to methods for increasing the percentage of muscle mass in a mammal, comprising administering an amount of aegeline effective to increase strength output. In some embodiments the mammal is a human. Accordingly, certain embodiments of the invention relate to methods for increasing strength output in a human comprising administering an effective amount of aegeline, and additional embodiments relate to methods for increasing the percentage of muscle mass in a human comprising administering an amount of aegeline effective to increase strength output in the human.

The present invention also relates to embodiments describing methods for affecting energy balance in a mammal (such as, e.g., a human), comprising administering an amount of aegeline, wherein the amount of aegeline results in an effect selected from the group consisting of increased mental stamina; increased thermogenesis; and increased strength output. In certain embodiments relating to affecting energy balance, the methods result in increased energy output over a unit of time, and in specific embodiments the unit of time ranges from about 4 hours to about 24 hours. In further embodiments, the methods result in an energy deficit over a unit of time, and in specific embodiments the unit of time ranges from about 4 hours to about 24 hours. In further embodiments, the present invention relates to methods for affecting body composition in a human, comprising administering an amount of aegeline effective to affect energy balance in the human. In some embodiments, the invention relates to methods for increasing the percentage of muscle mass in a human, comprising administering an amount of aegeline effective to increase thermogenesis in the human. In additional or alternative embodiments, the invention relates to methods for decreasing the percentage of fat mass in a human, comprising administering an amount of aegeline effective to increase thermogenesis in the human.

In addition, certain aspects of the present invention relate to a method of administering a food or dietary supplement comprising administering an amount of aegeline orally to a human, wherein the administering step results in one or more effects in the human selected from the group consisting of (a) an increase in mental stamina, (b) an increase in focus, (c) an increase in energy level, (d) an improvement in mood, (e) an increase in thermogenesis, and (f) an increase in muscle output. Further aspects of the invention relate to a method of administering an amount of aegeline orally to a human male, wherein the administering step results in an increase in libido in the human male.

Additional aspects of the invention relating to methods of administering aegeline comprise administering aegeline, wherein the administering step is repeated daily (or about daily) for at least two consecutive or non-consecutive days. In some embodiments, the methods comprise administering an amount of aegeline, wherein the amount of aegeline is a daily dose ranging from about 30 mg to about 3000 mg. In certain embodiments, the amount of aegeline is a daily dose ranging from about 40 mg to about 1000 mg. In specific embodiments, the amount of aegeline is a daily dose of about 40 mg or about 80 mg. In alternative embodiments, the amount of aegeline is a daily dose ranging from about 100 mg to about 200 mg, and in specific embodiments, the daily dose is about 120 mg. In other embodiments, the methods comprise administering an amount of aegeline, wherein the amount of aegeline is a daily dose ranging from about 100 mg to about 3000 mg. Accordingly, the amount of aegeline in some embodiments is a daily dose ranging from about 400 mg to about 1000 mg, and in further embodiments the amount of aegeline is a daily dose ranging from about 400 mg to about 800 mg. In specific embodiments, the daily dose is about 400 mg, about 600 mg, or about 800 mg.

Other aspects of the invention relating to methods of administering aegeline comprise administering aegeline, wherein the administering step is carried out twice per day. In alternative aspects, the administering step is carried out not more than once per day. In some embodiments, the administering step is repeated once every about ten hours to about twenty-four hours, over a time course ranging from about seven days to about six months. In specific embodiments, the amount of time between each administering step is not less than about twelve hours. Furthermore, some of the embodiments of the invention relate to methods of administering aegeline, wherein the amount of aegeline administered does not exceed about 1000 mg per about twenty-four hours.

Certain embodiments of the invention relate to methods of administering aegeline, wherein the administering step is repeated daily (or about daily) over a time course ranging from about seven days to about six months. In some embodiments, the amount of aegeline administered is a daily dose ranging from about 40 mg to about 800 mg. Additionally or alternatively, the daily dose is gradually increased from an initial daily dose up to a final daily dose. In some embodiments, the daily dose is increased by an amount ranging from about 10 mg to about 100 mg on a weekly, bi-monthly, or monthly basis. The invention also relates to embodiments wherein the daily dose is self-administered orally.

While some embodiments of the present invention relate to methods comprising repeated administrations of aegeline to a human, in certain of these embodiments, the invention relates to methods that result in a decreased percentage of fat mass in the human. Additionally or alternatively, in some embodiments, the methods result in an increased percentage of muscle mass in the human. Accordingly, some embodiments of the present invention relate to methods comprising administering aegeline to a human, wherein the methods result in a decreased percentage of fat mass and an increased percentage of muscle mass in the human. In certain embodiments, aegeline administration is carried out by ingesting an oral dosage form.

Some aspects of the present invention relate to methods comprising administering an amount of aegeline, wherein the amount of aegeline is an amount exhibiting an aegeline effect selected from the group consisting of (a) increased mental stamina, (b) increased energy level, (c) increased focus, (d) improved mood, (e) increased thermogenesis, and (f) increased strength output, wherein the aegeline effect is prolonged when compared to a caffeine effect, wherein the caffeine effect is selected from the group consisting of (a) increased mental stamina, (b) increased energy level, (c) increased focus, (d) improved mood, (e) increased thermogenesis, and (f) increased strength output, and wherein the caffeine effect results from administering an amount of caffeine effective to achieve the caffeine effect. In certain embodiments, the aegeline effect and the caffeine effect are together selected from the group consisting of (a) increased mental stamina, (b) increased energy level, (c) increased focus, (d) improved mood, (e) increased thermogenesis, and (f) increased strength output. In some embodiments, the aegeline effect is about 10% to about 70% longer in duration than the caffeine effect. In certain embodiments, the aegeline effect is about 20% to about 50% longer in duration than the caffeine effect. In other embodiments, the aegeline effect is about 200% to about 500% longer in duration than the caffeine effect. In specific embodiments, the aegeline effect and the caffeine effect are both an effect on mental stamina, and the aegeline effect is about 50% to about 300% longer in duration than the caffeine effect. In some embodiments, the aegeline effect on mental stamina is at least about 100% longer in duration compared to the caffeine effect on mental stamina.

Additional aspects of the present invention relate to methods of increasing mental stamina of a human comprising administering an effective amount of a composition, wherein the composition comprises aegeline, caffeine, bauhinia purpurea L. extract, hemerocallis fulva extract, yohimbe extract, and higenamine. Further aspects of the invention relate to methods of increasing focus of a human comprising administering an effective amount of a composition, wherein the composition comprises aegeline, caffeine, bauhinia purpurea L. extract, hemerocallis fulva extract, yohimbe extract, and higenamine. Embodiments of the invention also relate to methods of increasing energy level of a human comprising administering an effective amount of a composition comprising aegeline, caffeine, bauhinia purpurea L. extract, hemerocallis fulva extract, yohimbe extract, and higenamine, and some embodiments relate to methods of improving mood of a human comprising administering an effective amount of a composition comprising aegeline, caffeine, bauhinia purpurea L. extract, hemerocallis fulva extract, yohimbe extract, and higenamine.

In certain embodiments, the invention relates to methods of increasing thermogenesis in a human comprising administering an effective amount of a composition comprising aegeline, caffeine, bauhinia purpurea L. extract, hemerocallis fulva extract, yohimbe extract, and higenamine. Further embodiments relate to methods of increasing libido in a man comprising administering an effective amount of a composition comprising aegeline, caffeine, bauhinia purpurea L. extract, hemerocallis fulva extract, yohimbe extract, and higenamine. In additional embodiments, the methods of the invention relate to methods of increasing muscle output in a human comprising administering an effective amount of a composition comprising aegeline, caffeine, bauhinia purpurea L. extract, hemerocallis fulva extract, yohimbe extract, and higenamine. In alternative embodiments, the invention relates to methods of increasing thermogenesis in a human comprising administering an effective amount of a composition comprising aegeline and citicoline. Further embodiments relate to methods of increasing libido in a human male comprising administering an effective amount of a composition comprising aegeline and citicoline, and additional embodiments relate to methods of increasing muscle output in a human comprising administering an effective amount of a composition comprising aegeline and citicoline.

With respect to the various embodiments relating to methods comprising administering aegeline, in certain of these embodiments, the aegeline is administered orally. In some embodiments, the amount of aegeline administered is formulated to be administered orally. In addition, certain embodiments of the invention relate to methods comprising administering an amount of aegeline, wherein the amount of aegeline is administered as an oral dosage form selected from the group consisting of tablet, capsule, gel cap, powder, gum, gel, and solution. In some embodiments, the oral dosage form is a food or dietary supplement, and in further embodiments the food or dietary supplement also comprises caffeine, bauhinia purpurea L. extract, hemerocallis fulva extract, yohimbe extract, and higenamine. In additional or alternative embodiments, the composition comprises aegeline and citicoline. In addition, certain aspects of the invention relate to methods comprising administering aegeline, wherein the administering step is carried out by self-administering. In some embodiments, the aegeline is self-administered in the form of a food or dietary supplement.

Other aspects of the present invention relate to methods for synthesizing aegeline. In certain aspects, the methods for synthesizing aegeline comprise the steps of (a) preparing an amount of 2-amino-1-(4-methoxyphenyl)ethanone hydrochloride; (b) processing the amount of 2-amino-1-(4-methoxyphenyl)ethanone hydrochloride to form an amount of N-(2-(4-methoxyphenyl)-2-oxoethyl)cinnamamide; and (c) processing the amount of N-(2-(4-methoxyphenyl)-2-oxoethyl)cinnamamide to form an amount of aegeline.

In certain embodiments, preparing an amount of 2-amino-1-(4-methoxyphenyl)ethanone hydrochloride (step (a)) comprises reacting an amount of 1-(4-methoxyphenyl)ethanone with a halogenating reagent in the presence of heat. In further embodiments, the halogenating reagent is N-halosuccinimide, and in still further embodiments, the N-halosuccinimide is N-bromosuccinimide. In some embodiments, the reaction of an amount of 1-(4-methoxyphenyl)ethanone with a halogenating reagent is performed in the presence of an alcohol. In certain embodiments, the alcohol is a lower alcohol, and in specific embodiments the lower alcohol is ethanol. In addition, in certain embodiments this reaction of an amount of 1-(4-methoxyphenyl)ethanone with a halogenating reagent is performed at a temperature between about 35° C. and about 45° C. and for a time period of about 2.5 hours to about 3.5 hours. In specific embodiments, the temperature is about 40° C. and the time period is about 3 hours. In further embodiments, this reaction is followed by cooling to a temperature of about 25° C.

In additional embodiments, following cooling, the preparation of an amount of 2-amino-1-(4-methoxyphenyl)ethanone hydrochloride (step (a)) further comprises a dehalogenation/amination step, wherein the dehalogenation/amination step comprises adding hexamethylenetetramine. In certain embodiments, following the addition of hexamethylenetetramine, the reaction is incubated at a temperature of about 25° C. for a time period of about 3 hours. In further embodiments, the time period of about 3 hours is followed by adding concentrated hydrogen chloride, which is then followed by stirring at a temperature of about 25° C. for a time period of about 12 hours. Additional embodiments comprise, following the stirring, reducing the reaction volume and cooling to a temperature of about 5° C. Further embodiments comprise filtering and drying the amount of 2-amino-1-(4-methoxyphenyl)ethanone hydrochloride. In specific embodiments, the amount of 2-amino-1-(4-methoxyphenyl)ethanone hydrochloride corresponds to at least about 50% yield based on the amount of 1-(4-methoxyphenyl)ethanone.

In certain embodiments, processing the amount of 2-amino-1-(4-methoxyphenyl)ethanone hydrochloride to form an amount of N-(2-(4-methoxyphenyl)-2-oxoethyl)cinnamamide (step (b)) comprises reacting the amount of 2-amino-1-(4-methoxyphenyl)ethanone hydrochloride with cinnamoyl chloride in the presence of an organic base and an organic solvent. In some embodiments, the organic base is triethylamine, and in additional embodiments, the organic solvent is dichloromethane. The synthesis methods of the present invention also relate to embodiments wherein the reaction of the amount of 2-amino-1-(4-methoxyphenyl)ethanone hydrochloride with cinnamoyl chloride in the presence of an organic base and an organic solvent comprises stirring at a temperature of about 25° C. for time period of about 3 hours. In additional embodiments, the methods further comprise, after the time period of about 3 hours, adding water and cooling to a temperature of about 5° C. (optionally followed by washing and drying) to form a precipitate comprising the amount of N-(2-(4-methoxyphenyl)-2-oxoethyl)cinnamamide. In specific embodiments, the amount of N-(2-(4-methoxyphenyl)-2-oxoethyl)cinnamamide corresponds to at least about 40% yield based on the amount of 1-(4-methoxyphenyl)ethanone.

In certain embodiments of the synthesis methods, the processing of the amount of N-(2-(4-methoxyphenyl)-2-oxoethyl)cinnamamide to form an amount of aegeline (step (c)) comprises reacting the amount of N-(2-(4-methoxyphenyl)-2-oxoethyl)cinnamamide with a reducing agent. In some embodiments, the reducing agent is sodium borohydride. In additional embodiments, the reduction is performed in the presence of an alcohol. In specific embodiments, the alcohol is a lower alcohol, and in further embodiments the lower alcohol is ethanol. In some embodiments, the reduction is performed at temperature of about 10° C. for a time period of about 2 hours. In further embodiments, the reduction is followed by concentrating volume, adding water, and stirring at a temperature of about 5° C. to form a precipitated solid. Further embodiments comprise boiling the precipitated solid in alcohol and then cooling to a temperature of about 5° C. (optionally followed by washing and drying) to form the amount of aegeline. In specific embodiments of the synthesis methods, the amount of aegeline corresponds to at least about 30% yield based on the amount of 1-(4-methoxyphenyl)ethanone.

### DETAILED DESCRIPTION OF THE INVENTION

Certain aspects of the present invention relate to compositions, uses, and methods that exploit the unique and unexpected activity profile of aegeline. The compound aegeline occurs naturally in the plant Aegle marmelos Correa (commonly known as Bael). Govindachari, T. R. and Premila M. S., Some alkaloids from Aegle marmelos, Phytochem., 22:755-757 (1983); Riyanto et al., Alkaloids from Aegle marmelos (Rutaceae), Malaysian J. Anal. Sci., 7:463-465 (2001); Lanjhiyana et al., A validated HPTLC method for simultaneous estimation of two marker compounds in Aegle marmelos (L.) Corr., (Rutaceae) root bark, Der. Pharm. Lett., 4:92-97 (2012). The present invention shows that administration of aegeline to mammals can produce various desirable activities and effects such as, e.g., cognitive effects (such as increased mental stamina, increased focus, increased energy level, and improved mood), increased libido, increased thermogenesis, and anabolic effects (e.g., the ability for increased physical output or muscle-producing effects). The present invention further shows that the particular effect(s) achieved depends on the dose or amount of aegeline administered to the mammal, with relatively lower doses of aegeline affecting mental stamina, focus, energy level, and mood, with low- to mid-doses also affecting thermogenesis and libido, while comparatively higher doses also resulting in anabolic effects. In addition, unlike other compositions conventionally used to improve mental stamina and focus—compositions such as caffeine that typically produce only temporary or transient effects—administration of aegeline unexpectedly results in prolonged effects. These effects are improved in duration as well as in quality, as aegeline administration does not result in tremors, anxiety, nervousness, or other undesirable outcomes that can be associated with caffeine ingestion.

Accordingly, certain aspects of the present invention relate to nutritional products and food and dietary supplements comprising aegeline. Other aspects of the present invention relate to methods comprising the administration of aegeline to mammals. In certain aspects, the methods comprise administering aegeline to a mammal in order to increase or improve mental stamina, focus, energy level, and/or mood. In other aspects, the methods comprise administering aegeline to a mammal in order to increase thermogenesis (i.e., energy expenditure), influence energy balance, and/or affect body composition. In further aspects, the methods comprise administering aegeline to a mammal in order to increase or improve libido, and in still further aspects, the methods comprise administering aegeline to a mammal in order to increase anabolic effects and/or further affect body composition.

Certain aspects of the present invention relate to methods and uses of aegeline that achieve certain cognitive effects, including, for example, an increase or improvement in mental stamina, focus, energy level, and/or mood. An increase in mental stamina and/or focus may improve performance on cognitive tasks, including the ability to perform such tasks for longer durations of time. An increase or improvement in mental stamina and/or focus may also be described as an increase or improvement in alertness, concentration, and/or vigilance. Further, such effects may include changes or improvements in mood, vigor, and/or sense of well-being. Thus, certain aspects of the present invention relate to methods of administering an amount of aegeline effective to improve mood, sense of well-being, and/or vigor in a mammal. Certain embodiments of the present invention also relate to methods comprising administering to a mammal (such as, e.g., a human) an amount of aegeline effective to increase energy level, activeness, or a desire to engage in activities.

In similar aspects, an increase or improvement in mental stamina, focus, energy level, and/or mood may be associated with a decreased sense of fatigue, tiredness, or sluggishness. Thus in certain aspects, the amount of aegeline is effective to reduce feelings of distress, confusion, and/or sense of tiredness or fatigue in the mammal. Accordingly, certain embodiments of the present invention relate to methods comprising administering to a mammal (such as, e.g., a human) an amount of aegeline effective to increase mental stamina, energy level, and/or focus, and/or decrease feelings of fatigue, tiredness, and/or sluggishness.

Each of the aforementioned cognitive effects is encompassed in the methods and uses further described herein.

Additional aspects of the present invention relate to methods comprising the administration of an amount of aegeline effective to increase thermogenesis. Thermogenesis or energy expenditure can be assessed by measuring a mammal's level or rate of oxygen consumption and carbon dioxide production (for example, in a respiration chamber). In addition, observing or experiencing an increase in body temperature (e.g., feeling warm or hot) may indicate an increase in thermogenesis. Increasing thermogenesis is a mechanism by which aegeline administration may affect metabolic rate, energy balance, body composition, and/or fitness.

Accordingly, the present invention also relates to methods comprising the administration of an amount of aegeline effective to influence energy balance. Energy balance refers to energy input relative to energy output, and it may refer to an energy deficit or to an energy surplus. Energy balance may describe energy input relative to energy output over the course of hours, over the course of a day, or over the course of weeks or months. In humans, for example, energy input/output is commonly expressed as a 24-hour kilocalorie value, and a kilocalorie deficit or surplus is measured by subtracting the total amount of energy or kilocalories expended from the total amount of energy or kilocalories ingested during a given 24-hour time period. Administration of aegeline may affect energy balance by increasing energy output and/or decreasing energy input. Such effect(s) on energy balance can be helpful for weight management. In addition, such effect(s) on energy balance over time can lead to a change in body composition, such as an increase in the percentage of skeletal muscle mass and/or a decrease in the percentage of fat mass. Increasing energy output and/or decreasing energy input over time can therefore improve the health and fitness of a mammal.

Additional aspects of the present invention relate to methods comprising the administration of an amount of aegeline effective to increase or improve libido. The present invention shows that aegeline administration increases libido in male subjects, and that this effect can be persistent or prolonged without the need for frequent aegeline administrations.

Further aspects of the present invention concern aegeline administration to produce anabolic effects. Anabolic effects include an effect on muscle output, muscle mass, and/or body composition. Such effects may be described as the ability for intense physical output and/or an observable increase in the intensity of muscle contractions. Anabolic effects can also include an improvement in the ability to engage in intense physical activity with a reduced need for warm-up activity. Over time, anabolic effects may facilitate an increase in the percentage of muscle mass, including increased muscle fullness and density, which may optionally coincide with a reduction in the percentage of fat mass.

Administration of aegeline enhances exercise or physical performance, including strength output and/or performance of sustained or endurance-type activities. This outcome may be related to aegeline's ability to improve mental stamina, energy level, and/or focus, thereby prolonging the amount of time before an individual reaches a sense of fatigue. See, e.g., Noakes, T. D., Fatigue is a brain-derived emotion that regulates the exercise behavior to ensure the protection of whole body homeostasis, Frontiers Physiol. 3: 1-13 (2012). Accordingly, under certain embodiments, administration of aegeline to increase mental stamina, energy level, and/or focus, and optionally to produce a concomitant sense of lessened fatigue and exertion, may in turn increase force or total strength output when performing acute physical activity, and/or may improve or enhance performance for endurance-type activities. Additionally or alternatively, the enhancement of exercise or physical performance may relate to aegeline's ability to increase thermogenesis and consequently the amount of energy available for output. The effect may further relate to aegeline's anabolic effects. In embodiments where aegeline administration improves physical performance, continued use or administration of aegeline over time may lead to improved muscle strength and increased accrual of muscle mass, and/or a reduction in fat mass.

The present invention therefore additionally relates to methods of enhancing physical performance, including methods of improving endurance and methods of increasing strength output. One aspect of the present invention relates to uses and methods for providing energy and strengthening to skeletal muscles, and for facilitating the ability of skeletal muscles to sustain prolonged periods of intense physical activity, wherein the uses and methods comprise administering a composition or dietary supplement comprising aegeline. Another aspect of the present invention relates to uses and methods for increasing mental stamina, focus, energy level, and/or mood (and/or reducing the sense of fatigue), and/or increasing thermogenesis, and/or producing anabolic effects (and thereby increasing strength output), wherein the uses and methods comprise administering a composition or dietary supplement comprising aegeline.

Additional aspects of the present invention relate to repeated, extended, sustained, or prolonged administration of aegeline to a mammal. Such repeated, extended, or prolonged administration may occur over a time period ranging from days to months. Furthermore, repeated, extended, or prolonged administration may comprise daily (or about daily) administration of aegeline, or alternatively may comprise administration of aegeline at selected times, such as before engaging in or during tasks requiring mental stamina and focus, and/or before engaging in or during physical activity. Repeated, extended, sustained, or prolonged administration of aegeline may facilitate a gain in skeletal muscle strength and mass. Additionally or alternatively, repeated, extended, sustained, or prolonged administration of aegeline may facilitate a loss of fat mass. Furthermore, repeated, extended, sustained, or prolonged administration of aegeline may provide long-term improvements of a mammal's energy level, mood, and sense of well-being. Daily or about daily administration encompasses administrations that are repeated for at least two consecutive or non-consecutive days. In embodiments where administration is repeated for longer durations (e.g., for 7 days), such daily or about daily administration encompasses continuous administration (with no stops and starts) as well as intermittent administration (where administration stops and resumes at either regular or irregular intervals). For example, embodiments of the methods of the present invention relate to administration for one to six days per week, administrations in cycles (e.g., administrations that occur each day for 5 days, followed by 2 days of no administration; or administrations that occur for two to eight consecutive weeks, followed by a rest period of no administration for up to one week), and/or administration on alternate days.

Certain aspects of the present invention, therefore, relate to uses and methods for affecting body composition in a mammal, comprising administering to the mammal a composition or dietary supplement comprising aegeline. The present invention also relates to uses and methods for promoting weight management in a human, comprising administering to the human a composition or dietary supplement comprising aegeline. In humans, a change in body composition that includes an increase in percentage muscle mass and/or a decrease in percentage fat mass generally improves an individual's health and fitness, and such a change in body composition is particularly beneficial to individuals for whom weight loss or a reduction in fat mass is necessary to combat health risks associated with obesity. In addition, athletes and fitness professionals will benefit from methods for altering body composition as described herein.

As used throughout the description, the terms administration, administering, administer, etc. encompass any form of administration, including but not limited to self-administration. In addition, unless stated otherwise, administering an amount of aegeline, or a composition comprising aegeline, to a mammal encompasses administering to human, bovine, equine, ovine, hircine, porcine, canine, feline, etc.

Other aspects of the present invention relate to compositions and methods for increasing or improving mental stamina, focus, energy level and/or mood, increasing thermogenesis (and/or affecting energy balance), increasing libido, and/or increasing anabolic effects, wherein the compositions and methods are directed to a naturally-occurring compound that exhibits an improved activity profile compared to other agents, including but not limited to caffeine. For example, with respect to cognitive effects, the present invention shows that aegeline exhibits unexpected and advantageous properties, including when compared to other agents taken for their effects on mental stamina and focus. The present invention demonstrates that aegeline administration unexpectedly produces a prolonged increase or improvement in mental stamina, focus, energy level, and/or mood when compared to the more temporary or transient effects on mental stamina, focus, energy level, and/or mood that are associated with conventional compositions (e.g., caffeine). Aegeline unexpectedly produces effects of longer duration compared to these other conventional compositions. Aegeline's enduring effects reduce the need for frequent administrations and eliminate the undesirable “crash,” or rapid dissipation of effects, associated with other agents. The unexpected and improved activity profile of aegeline therefore allows methods and uses comprising less frequent doses and/or longer durations of effectiveness when compared to methods and uses involving other compositions such as, e.g., caffeine. Accordingly, certain aspects of the present invention relate to uses and methods relating to the uniquely prolonged half-life of aegeline's effect(s).

In some embodiments, the increased or improved mental stamina, focus, energy level, and/or mood, increased thermogenesis, increased libido, and/or increased anabolic effects resulting from aegeline ingestion continue(s) for a substantial period of time. For example, the duration of time may last anywhere between about 2 hours to about 30 hours. In further embodiments, the increased or improved mental stamina, focus, energy level, and/or mood, increased thermogenesis, increased libido, and/or increased anabolic effects resulting from aegeline ingestion continue(s) for a duration of time lasting anywhere between about 4 hours to about 24 hours. In still further embodiments, the increased or improved mental stamina, focus, energy level, and/or mood, increased thermogenesis, increased libido, and/or increased anabolic effects resulting from aegeline ingestion continue(s) for a duration of time lasting anywhere between about 4 to about 20 hours. The increased or improved mental stamina, focus, energy level, and/or mood, increased thermogenesis, increased libido, and/or increased anabolic effects may endure for a time period that is anywhere between about 2 to about 10 hours, about 2 to about 8 hours, about 2 to about 6 hours, about 2 to about 4 hours, or about 2 to about 3 hours. In specific embodiments, the increased or improved mental stamina, focus, energy level, and/or mood, increased thermogenesis, increased libido, and/or increased anabolic effects resulting from aegeline ingestion continue(s) for a duration of time lasting for about 2 hours, about 3 hours, about 4 hours, about 5 hours, about 6 hours, about 8 hours, about 10 hours, or about 12 hours. In other specific embodiments, the increased or improved mental stamina, focus, energy level, and/or mood, increased thermogenesis, increased libido, and/or increased anabolic effects resulting from aegeline ingestion continue(s) for a duration of time lasting for about 14 hours, about 16 hours, about 18 hours, about 20 hours, about 24 hours, or about 30 hours. In additional embodiments, the increased or improved mental stamina, focus, energy level, and/or mood, increased thermogenesis, increased libido, and/or increased anabolic effects resulting from aegeline ingestion endure(s) until the next subsequent administration of aegeline. In further embodiments, the next subsequent administration of aegeline occurs on the following day.

In the latter embodiments, an effect that is prolonged, extended, continued, enduring, etc., and similarly an effect that continues, endures, etc., includes an effect for which the degree or extent of the effect is not constant or unvarying during the time within which it continues or endures. For example, an increase or improvement in mental stamina, focus, energy level, and/or mood resulting from aegeline administration that continues or endures for about 10 hours includes an increase or improvement in mental stamina, focus, energy level, and/or mood whereby the increase or improvement varies in degree during the about 10 hours. In this example, such variation would include an effect that is greater in degree during the first about 5 hours following effect onset compared to the effect's degree during the second about 5 hours.

Accordingly, in certain aspects of the present invention, the amount of aegeline administered exhibits an effect of increased or improved mental stamina, focus, energy level, and/or mood, increased thermogenesis, increased libido, and/or increased anabolic effects, wherein the effect is at least about 15% to about 500% longer in duration compared to a corresponding effect observed following administration of conventional composition(s) (such as, for example, caffeine) used to achieve such effects. In further aspects, the effect is at least about 15%, about 20%, about 25%, about 30%, about 40%, about 50%, about 60%, about 70%, about 80%, about 90%, about 100%, about 150%, about 200%, or about 300% longer in duration compared to a corresponding effect observed following administration of conventional compositions used to achieve such effects. In addition, certain embodiments of the present invention relate to methods comprising administering aegeline to achieve an effect selected from the group consisting of increased or improved mental stamina, increased focus, increased energy level, improved mood, increased thermogenesis, increased libido, and increased anabolic effects, wherein the effect achieved is of longer duration compared to such effect achieved by analogous methods comprising administering a composition without aegeline. In certain embodiments, the effect achieved or resulting from a method comprising aegeline administration endures or continues for a period of time that is at least about 15%, about 20%, about 25%, about 30%, about 40%, about 50%, about 60%, about 70%, about 80%, about 90%, about 100%, about 150%, about 200%, or about 300% longer than the duration of effect achieved by analogous methods comprising administering a composition without aegeline. A composition without aegeline includes, but is not limited to, a composition comprising caffeine but not aegeline.

The present invention also relates to methods comprising administering aegeline to achieve increased mental stamina, increased focus, increased energy level, improved mood, increased thermogenesis, and/or increased libido, whereby any associated troughs or “crashes” (i.e., rapid dissipation in effects) are unexpectedly reduced when compared to troughs and crashes resulting from a method comprising administering a composition conventionally used to increase mental stamina, increase focus, increase energy level, improve mood, increase thermogenesis, and/or increase libido. Compositions that are conventionally used for such purposes may include, for example, caffeine for effects on mental stamina, focus, energy level, mood, and/or thermogenesis.

These aspects of the present invention further relate to methods that achieve in mammals an effect on mental stamina, focus, energy level, and/or mood, and optionally on thermogenesis, libido, and/or anabolic effects, to a greater extent relative to other methods using conventional agents (such as caffeine for certain effects), at least in part because aegeline's effect(s) exhibit longer duration per each administration compared to the effect(s) of conventional agents. These aspects of the present invention, therefore, relate to methods comprising administering aegeline to humans to achieve an effect on mental stamina, focus, energy level, mood, thermogenesis, libido, and/or anabolic effects, whereby the effect(s) achieved are greater than such effect(s) achieved by analogous methods comprising administering compositions without aegeline. An analogous method may include a method employing the same number and frequency of dosings or administrations. Similarly, the present invention relates to methods comprising administering aegeline to a mammal to increase or improve mental stamina, focus, energy level, mood, thermogenesis, libido, and/or percentage muscle mass, wherein the increase or improvement achieved is at least substantially or about the same as an increase or improvement achieved by a method comprising administering a composition without aegeline, but wherein the number and/or frequency of aegeline administrations is less than the number and/or frequency of administrations of the composition without aegeline.

The present invention also shows that, in addition to improved duration, aegeline's effects unexpectedly exhibit improved quality. For example, the administration of aegeline to increase mental stamina, focus, energy level, and/or improve mood does not produce anxiety, tremors, or other unwanted behavioral effects, all of which are effects that have been reported following caffeine administration. Aegeline administration also eliminates the crash, or rapid dissipation of effect, that is typically experienced with caffeine administration. Furthermore, ceasing aegeline administration does not result in the withdrawal symptoms that are typically associated with the termination of caffeine ingestion. Thus, aegeline administration exhibits unexpected and advantageous properties, including with respect to the quality and nature of cognitive effects, when compared to conventional agents such as caffeine. Aegeline's effects on mental stamina, focus, energy level, and mood are improved in quality when compared to similar effects of other substances such as caffeine.

The present invention also demonstrates that aegeline unexpectedly exhibits different activities depending on the doses administered. The unexpected dose-dependent activity profile of aegeline includes increased or improved mental stamina, focus, energy level, and/or mood when aegeline is ingested at relatively lower doses or amounts. When such relatively lower doses or amounts are increased, aegeline's effect profile additionally includes increased thermogenesis and increased libido. Furthermore, when aegeline is ingested at relatively higher doses or amounts, the effect profile also includes increased anabolic effects, which may be observed as improved physical performance and strength output, and/or an increase in percentage muscle mass and/or a decrease in percentage fat mass. Certain studies have examined aegeline's effects but have not reported these unexpected and advantageous differential dose effects. See Narender et al., Antihyperglycemic and antidyslipidemic agent from Aegle marmelos, Bioorg. Med. Chem. Lett. 17: 1808-11 (2007); Nugroho et al., Effects of aegeline, a main alkaloid of Aegle marmelos correa leaves, on the histamine release from mast cells, Pak. J. Pharm. Sci., 24: 359-367 (2011).

Thus, certain aspects of the present invention relate to methods and uses comprising administering aegeline in an amount effective to increase or improve mental stamina, focus, energy level, and/or mood, but not to produce anabolic effects or an increase in muscle mass. Other embodiments of the present invention relate to methods comprising administering aegeline to increase mental stamina, focus, energy level, and/or improve mood, wherein the amount or dose of aegeline administered is less than an amount or dose of aegeline that increases thermogenesis and/or affects energy balance, and/or produces anabolic effects. Another aspect of the present invention relates to methods comprising administering aegeline to affect mental stamina, focus, energy level, and/or mood, without significantly affecting thermogenesis or producing anabolic effects. In still further embodiments, the methods result in one or more of the aforementioned cognitive or mood effects without significantly affecting or increasing libido.

Thus, in certain embodiments, the amount of aegeline administered to increase thermogenesis (and/or affect energy balance), increase libido, and/or produce anabolic effects (and/or affect body composition), is about 2 times to 50 times greater than the amount of aegeline administered to increase or improve mental stamina, focus, energy level, and/or mood. In further embodiments, the amount of aegeline administered to increase thermogenesis (and/or affect energy balance), increase libido, and/or produce anabolic effects (and/or affect body composition) is about 2 times to 20 times greater than the amount of aegeline administered to increase or improve mental stamina, focus, energy level, and/or mood. In specific embodiments, the amount of aegeline administered to increase thermogenesis (and/or affect energy balance), increase libido, and/or produce anabolic effects (and/or affect body composition) is about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, or about 15 times greater than the amount of aegeline administered to increase or improve mental stamina, focus, energy level, and/or mood.

In alternative embodiments, the present invention relates to methods and uses comprising administering aegeline in an amount effective to increase or improve mental stamina, focus, energy level, and/or mood, and to increase thermogenesis, but not to produce anabolic effects or an increase in muscle mass. Such embodiments of the present invention relate to methods comprising administering aegeline to increase mental stamina, focus, energy level, and/or improve mood, and/or to increase thermogenesis, wherein the amount or dose of aegeline administered is less than an amount or dose of aegeline effective to produce anabolic effects. Another aspect of the present invention relates to methods comprising administering aegeline to affect mental stamina, focus, energy level, and/or mood, and/or to affect thermogenesis, without significantly producing anabolic effects. In certain embodiments, the methods result in one or more of the aforementioned cognitive or mood effects while also increasing thermogenesis, but without increasing or significantly affecting libido. In alternative embodiments, the methods result in one or more of the aforementioned cognitive or mood effects while also increasing thermogenesis and increasing libido, but without significantly affecting anabolic effects. In further of these embodiments, the methods result in one or more of the aforementioned cognitive or mood effects while also increasing thermogenesis and increasing libido, but without significantly producing anabolic effects.

By not significantly producing anabolic effect(s), such methods may result in a subtle anabolic effect that is noticeable only after prolonged aegeline administration (e.g., daily administration of aegeline for at least about 14 days). These methods that do not significantly produce anabolic effect(s) may be contrasted to methods comprising administration of aegeline at higher daily doses, where an anabolic effect is apparent more immediately.

Thus, in certain embodiments, the amount of aegeline administered to produce anabolic effects (and/or affect body composition) is about 2 times to 40 times greater than the amount of aegeline administered to increase libido, increase thermogenesis (and/or affect energy balance), and/or increase or improve mental stamina, focus, energy level, and/or mood. In further embodiments, the amount of aegeline administered to produce anabolic effects (and/or affect body composition) is about 2 times to 20 times greater than the amount of aegeline administered to increase libido, increase thermogenesis (and/or affect energy balance), and/or increase or improve mental stamina, focus, energy level, and/or mood. In specific embodiments, the amount of aegeline administered to produce anabolic effects (and/or affect body composition) is about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, or about 15 times greater than the amount of aegeline administered to increase libido, increase thermogenesis (and/or affect energy balance), and/or increase or improve mental stamina, focus, energy level, and/or mood.

As indicated in the embodiments above, the doses or amounts of aegeline ingestion can be selected to produce a particular effect profile. The present invention provides the dose ranges sufficient for each effect.

Aegeline occurs naturally in the bael plant, or Aegle marmelos Correa, and can be extracted from the plant's leaves, root bark, and fruit. See Borde et al., Extraction and thin layer chromatography of alkaloids from Bael (Aegle marmelos) leaves, J. Ecobiotech. 3: 1-4 (2011); Faizi et al., Shahidine, a novel and highly labile oxazoline from Aegle marmelos: the parent compound of aegeline and related amides, Tetrahedron 65: 998-1004 (2009); Riyanto et al., Alkaloids from Aegle marmelos (Rutaceae), Malaysian J. Anal. Sci. 7: 463-65 (2001); Patel, P. K. et al., Aegle marmelos: A review of its medicinal properties, Internat. J. Pharm. Phytopharm. Res., 1: 332-41 (2012), the disclosures of which are incorporated herein by reference. One study also detected aegeline in an extract from Solanum erianthum (Solanaceae), a plant which grows in Nigeria. Ali et al., Naturally Occurring Antifungal Aromatic Esters and Amides, J. Chem. Soc. Pak. (32): 565-569 (2010). In addition to its natural occurrence, aegeline can be made synthetically using various synthesis methods. See Aguirre et al., Asymmetric synthesis of naturally occurring β-hydroxyamides (R)-tembamide and (R)-aegeline, Rev. Soc. Quim. Méx., 45: 21-24 (2001); Cho et al., Application of optically active 1,2-diol monotosylates for synthesis of β-azido and β-amino alcohols with very high enantiomeric purity. Synthesis of enantiopure (R)-octopamine, (R)-tembamide and (R)-aegeline, Tetrahedron: Asymmetry 13: 1209-17 (2002); Sadyandy et al., An asymmetric dihydroxylation route to (R)-(−)-octopamine, (R)-(−)-tembamide and (R)-(−)-aegeline, Arkivoc 3: 36-43 (2005); Lee et al., Asymmetric transfer hydrogenation of 2-tosyloxy-1-(4-hydroxyphenyl)ethanone derivatives: synthesis of (R)-tembamide, (R)-aegeline, (R)-octopamine, and (R)-denopamine, Tetrahedron: Asymmetry 18: 2662-67 (2007); Fardelone et al., Chiral pharmaceutical intermediaries obtained by reduction of 2-halo-1-(4-substituded phenyl)-ethanones mediated by Beotrichum candidum CCT 1205 and Rhodotorula glutinis CCT 2182, Enzyme Res. (2011): 1-8; the disclosures of which are incorporated herein by reference. Thus, in certain embodiments, aegeline is extracted, isolated, and/or purified from natural sources; in other embodiments, aegeline is made synthetically.

Aegeline, the chemical structure of which is shown below, is a chiral β-hydroxyamide. Thus, other terms for aegeline include (R)-aegeline and (S)-aegeline, and 2-Propenamide, N-[(2R)-2-hydroxy-2-(4-methoxyphenyl)ethyl]-3-phenyl-, (2E)- and 2-Propenamide, N-[(2S)-2-hydroxy-2-(4-methoxyphenyl)ethyl]-3-phenyl-, (2E).

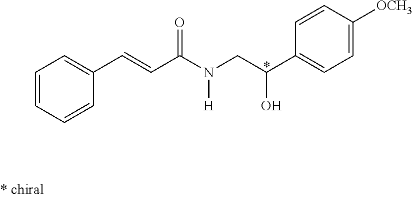 [SMILES: COc1ccc(C(O)CNC(=O)/C=C/c2ccccc2)cc1]

Accordingly, unless indicated otherwise, the term aegeline and its synonyms as used herein encompass all stereoisomers and enantiomers of aegeline and mixtures of these isomeric forms. These enantiomeric forms include (R)-aegeline and (S)-aegeline. See Sadyandy et al. (2005); Albonico et al., Tembamide from Fagara hyemalis (St. Hill.) Engler, J. Chem. Soc. C, 1967, 1327-1328; Kamal et al., Chemoenzymatic synthesis of (R)- and (S)-tembamide, aegeline and denopamine by a one-pot lipase resolution protocol, Tetrahedron: Asymmetry (15), 3939-3944; Fardelone et al., Bioreduction of 2-azido-1-arylethanones mediated by Geotrichum candidum and Rhodotorula glutinis, J. Molecular catalysis B: Enzymatic (39): 9-12 (2006); Yadav et al., Stereoselective synthesis of (R)-(−)-denopamine, (R)-(−)-tembamide, and (R)-(−)-aegeline via asymmetric reduction of azidoketones by Daucus carota in aqueous medium, ChemInform 33: 2002; Brown et al., Synthetic applications of optically active cyanohydrins, enantioselective syntheses of the hydroxyamides tembamide (VIa) and aegeline (VIb), the cardiac drug denopamine (XI), and some analogues of the Bronchodilator Salbutamol (XIIa). ChemInform 26 (1995); and Fardelone et al. (2011), the disclosures of which are all incorporated herein by reference.

As described herein, unless otherwise indicated, the term aegeline and its synonyms encompass the racemic mixture of (R)-aegeline and (S)-aegeline, either enantiomer in its pure or substantially pure form (for example, (R)-aegeline substantially free from (S)-aegeline, or (S)-aegeline substantially free from (R)-aegeline), and the compound as a non-racemic or enantiomerically enriched form (i.e., a mixture having greater than 50% but less than 100% of either enantiomer, and less than 50% of the other enantiomer). In some embodiments of the invention, an enriched mixture of a given enantiomer contains no more than about 40%, 35%, 30%, 25%, 20%, 15%, 10%, 5%, 4%, 3%, 2%, or 1% of the other enantiomer. In particular embodiments of the present invention, the enriched aegeline form contains mainly (R)-aegeline and no more than about 40%, 35%, 30%, 25%, 20%, 15%, 10%, 5%, 4%, 3%, 2%, or 1% of the (S)-aegeline enantiomer. In one embodiment, the enantiomer administered is (R)-aegeline in its pure or substantially pure form. In alternative embodiments, the enriched aegeline form contains mainly (S)-aegeline and no more than about 40%, 35%, 30%, 25%, 20%, 15%, 10%, 5%, 4%, 3%, 2%, or 1% of the (R)-aegeline enantiomer. Furthermore, in particular embodiments, the aegeline is enantiomerically enriched (R)-aegeline. In a further embodiment, the aegeline is pure or substantially pure (R)-aegeline.

Stereoisomeric purity of compounds may be established by conventional analytical methods well known to those of skill in the art. For example, use of chiral NMR shift reagents, gas chromatographic analysis using chiral columns, high pressure liquid chromatographic analysis using chiral columns, polarimetry, isotopic dilution, calorimetry, enzymatic methods, capillary electrophoresis on chiral gels, formation of diastereomeric derivatives through reaction with chiral reagents and conventional analysis via known analytical methods may be used to establish the stereochemical purity of a specific stereoisomer. Alternatively, synthesis using starting materials of known stereochemical enrichment may be used to establish the stereochemical purity of the compounds described herein. Other analytical methods for demonstrating stereochemical homogeneity are known in the field. See, e.g., Organic Syntheses, Coll., 49: 93 (1969) and 5: 932 (1973); Theilacker and Winkler, Chemische Berichte, 87: 690-91 (1954); Ferreira et al., Rational approach to the selection of conditions for diastereomeric resolution of chiral amines by diacid resolving agents, Tetr. Asymmetry 17: 1337-1348 (2006); the disclosures of which are all incorporated herein by reference.

Additional aspects of the present invention, therefore, relate to uses and methods comprising administering non-racemic (i.e., optically active, or where the ratio of enantiomers is not 1:1) aegeline to increase or improve mental stamina, focus, energy level, and/or mood, increase thermogenesis, increase libido, and/or produce anabolic effects or affect body composition. Accordingly, certain embodiments of the present invention relate to methods comprising administering an enriched aegeline form to humans, where the enriched forms contains mainly (R)-aegeline and no more than about 40%, 35%, 30%, 25%, 20%, 15%, 10%, 5%, 4%, 3%, 2%, or 1% of the (S)-aegeline enantiomer. In one embodiment, the method comprises administration of pure or substantially pure (R)-aegeline. In alternative embodiments, the enriched aegeline form contains mainly (S)-aegeline and no more than about 40%, 35%, 30%, 25%, 20%, 15%, 10%, 5%, 4%, 3%, 2%, or 1% of the (R)-aegeline enantiomer.

The present invention encompasses all aegeline compounds, including the base compound and any salts and esters thereof, administered by oral, transdermal, intranasal, or other parenteral route in mammals (e.g., in humans). Where stated, the present invention also relates to precursors and/or derivatives that yield the compound before or after oral, transdermal, intranasal, or other parenteral administration in mammals (e.g., in humans). See, e.g., Shahidine et al. (2009).

Accordingly, the terms aegeline and its synonyms, and references to the compound's enantiomers, are meant to encompass all salts of the compound. A salt of aegeline refers to the base compound and derivatives of aegeline that are modified by making acid salts thereof. Such salts include, but are not limited to, mineral or organic acid salts, and the conventional non-toxic salts or the quaternary ammonium salts, for example, from non-toxic inorganic or organic acids. Such conventional non-toxic salts include those derived from inorganic acids such as hydrochloric acid, hydrobromic acid, sulfuric acid, sulfamic acid, phosphoric acid, and nitric acid; and the salts prepared from organic acids such as formic acid, acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid, methanesulfonic acid, ethane disulfonic acid, benzenesulfonic acid, oxalic acid, isethionic acid, acidic amino acids (such as aspartic acid and glutamic acid), and the like. In some embodiments, therefore, aegeline salts of the present invention include, for example, aegeline acetate, hydrochloride, ethyl ester, tartrate, malate, lactate, sulfate, maleate, fumarate, citrate, oxaloacetate, pyruvate, etc. Certain embodiments of the present invention relate to aegeline succinate.

Salts of the present invention can be synthesized by reacting the free base of aegeline with a stoichiometric amount of the appropriate acid in water or in an organic solvent, or in a mixture of the two—generally, nonaqueous media such as ether, ethyl acetate, ethanol, isopropanol, or acetonitrile may be used. Lists of exemplary salts are found in texts such as Remington's Pharmaceutical Sciences, 18th ed., Mack Publishing Company, Easton, Pa., 1990, which is incorporated herein by reference.

In certain aspects of the present invention, aegeline is administered to an individual in an amount sufficient for the individual to experience increased or improved mental stamina, focus, energy level, and/or mood, and optionally, as indicated above, decreased sense of fatigue, tiredness, and/or sluggishness. In certain embodiments of the present invention, aegeline is administered to a human in an amount effective or sufficient to increase thermogenesis (which may, in turn, affect energy balance), and/or produce anabolic effects (which may enable increased physical output and/or affect body composition and increase percentage muscle mass). Certain of these embodiments may also result in increased libido. In certain embodiments, aegeline is administered in an amount of about 40 mg to about 3000 mg. For example, a preferred amount of aegeline may be an amount of about 40 mg to about 1000 mg. In particular embodiments, aegeline is administered in an amount of about 40 mg, about 80 mg, or about 120 mg. In other embodiments, aegeline is administered in an amount of about 50 mg, 75 mg, 100 mg, 125 mg, 150 mg, 175 mg, 200 mg, 225 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1250 mg, 1500 mg, 1750 mg, 2000 mg, 2250 mg, 2500 mg, 2750 mg, or 3000 mg. In some embodiments, the amount of aegeline administered is an amount per kilogram (kg). In certain aspects of such embodiments, the amount ranges from about 0.75 mg/kg to about 50 mg/kg, and in preferred aspects the amount ranges from about 0.75 mg/kg to about 20 mg/kg. In further embodiments, the amount of aegeline is about 0.75 mg/kg, 1 mg/kg, 1.5 mg/kg, or 2 mg/kg. In other aspects, the amount of aegeline is about 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 m/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 16.5 mg/kg, 17 mg/kg, 17.5 mg/kg, 18 mg/kg, 18.5 mg/kg, 19 mg/kg, 19.5 mg/kg, or 20 mg/kg.

Accordingly, certain aspects of the present invention relate to methods and uses comprising administering an amount of aegeline effective to increase or improve mental stamina, focus, energy level, and/or mood, wherein the amount of aegeline is an amount ranging from about 40 mg to about 3000 mg. For example, a preferred amount of aegeline may be an amount ranging from about 40 mg to about 1000 mg. In particular embodiments, aegeline is administered in an amount of about 40 mg, about 80 mg, or about 120 mg. In certain embodiments, an amount of aegeline effective to increase or improve mental stamina, focus, energy level, and/or mood is an amount ranging from about 60 mg to about 1000 mg. In further embodiments, an amount of aegeline effective to increase or improve mental stamina, focus, energy level, and/or mood is an amount ranging from about 60 mg to about 200 mg. In certain embodiments, such an amount of aegeline is about 80 mg, about 100 mg, about 120 mg, about 125 mg, about 150 mg, about 175 mg, or about 200 mg. In specific embodiments, this amount is administered once per day. In similar embodiments, the amount of aegeline effective to increase or improve mental stamina, focus, energy level, and/or mood is an amount ranging from about 0.5 mg/kg to about 5 mg/kg. In further embodiments, such an amount of aegeline is about 0.7 mg/kg, 1 mg/kg, 1.5 mg, kg, 2 mg/kg, 2.5 mg/kg, 3 mg/kg, 3.5 mg/kg, 4 mg/kg, or 4.5 mg/kg.

Other embodiments of the present invention relate to methods and uses comprising administering an amount of aegeline effective to increase thermogenesis, increase libido, and/or produce anabolic effects (e.g., improve physical performance and/or increase muscle or strength output), wherein the amount of aegeline is an amount ranging from about 100 mg to about 3000 mg. In certain embodiments, the amount of aegeline is an amount ranging from about 120 mg to about 3000 mg. In further embodiments, the amount ranges from about 200 mg to about 1000 mg. In particular embodiments, the amount of aegeline is about 120 mg, about 200 mg, about 300 mg, about 400 mg, about 500 mg, about 600 mg, about 700 mg, about 800 mg, about 900 mg, or about 1000 mg. In specific embodiments, this amount is administered once per day. In similar embodiments, the amount of aegeline effective to increase thermogenesis, increase libido, and/or produce anabolic effects (e.g., improve physical performance and/or increase muscle or strength output), is an amount ranging from about 2 mg/kg to about 50 mg/kg. In further embodiments, the amount ranges from about 5 mg/kg to about 40 mg/kg, and in still further embodiments the amount ranges from about 10 mg/kg to about 40 mg/kg. In particular embodiments, the amount of aegeline is about 5 mg/kg, 10 mg/kg, 15 mg/kg, 20 mg/kg, 25 mg/kg, 30 mg/kg, 35 mg/kg, 40 mg/kg, or 45 mg/kg. In these embodiments, repeat administrations of aegeline can result in a change in body composition, including, for example, an increase in percentage muscle mass and/or a decrease in percentage fat mass.

In certain embodiments, the amount of aegeline is administered as a daily dose. In addition, a daily dose may be administered as a single daily dose, or alternatively may be divided into multiple doses to be taken throughout the day (for example, in two administrations (e.g., morning and afternoon), in three administrations (e.g., morning, mid-day, and late afternoon/evening), etc.). A given amount or daily dose may depend on a variety of factors, including, for example, the individual's size, sex, medical history, health status, and/or other agents or medications taken. Furthermore, a given amount or daily dose of aegeline may depend on whether the compound is administered with other agents, and/or whether it is administered as a racemate or as an enantiomerically enriched or enantiomerically pure form.

In some embodiments, the compound aegeline can be combined with another compound or component as part of a formulation or supplement. Accordingly, the present invention relates to compositions comprising aegeline in combination with one or more other substances, agents, components, compounds, or dietary ingredients. Similarly, as used throughout the description, methods comprising administering aegeline encompass combination treatments wherein the compound is administered with one or more other substances, compounds, agents, supplements, dietary ingredients, etc. Such other substances, compounds, agents, supplements, dietary ingredients, etc., include but are not limited to amino acids, minerals, vitamins, and more generally antioxidants, nutrients, and plant extracts or other naturally-occurring compounds. These substances include, without limitation, caffeine, theophylline, paraxanthine, phenethylamine, arginine α-ketoglutarate, nicotinamide, riboflavin, pantothenic acid, propionyl L-carnitine, tyrosine, n-acetyl-l-tyrosine, creatine (in any form, including, e.g., creatine monohydrate and creatine ethyl ester), δ-alanine, caffeic acid, citicoline, rauwolscine (e.g., rauwolscine HCL), yohimbine (including yohimbe (pausinystalia johimbe) (bark) extract), bauhinia purpurea L. extract, hemerocallis fulva extract, higenamine (or norcoclaurine), cirsium oligophyllum extract, bacopa monnieri extract, schisandra chinensis extract, and other naturally occurring compounds or plant extracts. Such other substances also include, without limitation, vitamin-like substances such as coenzyme Q₁₀.

Accordingly, formulations or combinations comprising aegeline include but are not limited to the following: a composition comprising aegeline and one or more other naturally occurring substances (including, e.g., a plant extract); a composition comprising aegeline and caffeine; a composition comprising aegeline and one or more other naturally occurring substances selected from the group consisting of caffeine, citicoline, bauhinia purpurea L. extract, hemerocallis fulva (flower) extract, yohimbe (pausinystalia johimbe) (bark) extract, and higenamine (or norcoclaurine). The formulations and combinations of the invention therefore include but are not limited to a composition comprising aegeline, caffeine, and one or more other naturally occurring substances. In further embodiments, such naturally occurring substance is selected from the group consisting of bauhinia purpurea L. extract, hemerocallis fulva (flower) extract, and yohimbe (pausinystalia johimbe) (bark) extract and norcoclaurine hydrochloride. As used herein, a naturally occurring substance includes compounds and substances isolated or extracted from plant material, as well as compounds and substances that occur naturally but that are made synthetically.

Compositions comprising aegeline, including combinations comprising aegeline, may be formulated for oral administration. Accordingly, in certain embodiments of the present invention, aegeline is formulated as a solid or liquid oral dosage form, such as, for example, tablet, capsule, gel cap, powder, gel, paste, solution or other orally ingestible form. In the above and other combinations, administration may involve the administration of a single composition that in turn comprises multiple components or substances, including aegeline. Such an example would be a dietary supplement, formulated as an oral dosage form (e.g., tablet, capsule, gel, powder, or solution), that comprises several dietary ingredients and optionally other ingredients (including, e.g., excipients). In addition or alternatively, methods comprising administering aegeline may involve concurrent administration of multiple, separate oral dosage forms, or any other combination of administrations and dosage routes.

The formulations and compositions of the present invention may optionally include one or more other ingredients, including, for example, one or more excipients. Such other ingredients include but are not limited to binders (e.g., gelatin, saccharides, polymers, cellulose, starch, etc.), disintegrants (e.g., crosslinked polymers, modified starch, colloidal silicon dioxide, etc.), fillers (e.g., vegetable oils, lactose, sucrose, glucose, mannitol, sorbitol, magnesium stearate, etc.), lubricants and glidants (e.g., talc, magnesium stearate, silicon dioxide, etc.), stabilizers (e.g., modified food starch, gelatin, etc.), as well as flavors, colorings, and coating agents. Thus, according to certain embodiments, the present invention relates to compositions comprising aegeline, caffeine, and one or more other naturally occurring substances, wherein the naturally occurring substance is selected from the group consisting of bauhinia purpurea L. extract, hemerocallis fulva (flower) extract, yohimbe (pausinystalia johimbe (bark) extract, and higenamine or norcoclaurine, and wherein the composition further comprises one or more other ingredients (such as, e.g., one or more excipients). In other embodiments, the composition comprises aegeline and citicoline and further comprises one or more other ingredients (such as, e.g., one or more excipients). In specific embodiments, the one or more other ingredients is selected from the group consisting of gelatin, modified starch or maltodextrin, vegetable or magnesium stearate, silicon dioxide, and artificial or natural food colorings.

Certain embodiments of the present invention further relate to combinations and formulations comprising aegeline and one or more other dietary and/or other ingredients in specific proportions. For example, an amount of aegeline in a given composition may range from about 10% to about 20% of the composition by weight, and such compositions may include one or more dietary or other ingredients, including, for example, caffeine, one or more other naturally occurring substances, and/or one or more other ingredients (e.g., one or more excipients). Possible proportions (or percentages by weight) for these dietary and other ingredients include, for example, about 30% to about 60% caffeine; about 20% to about 50% of one or more other naturally occurring substance(s); and about 5% to about 15% excipient(s). A more specific example under this embodiment may include the following ranges or proportions: aegeline (about 10% to about 15%); caffeine (about 35% to about 45%); one or more plant extracts or other naturally occurring substances (about 25% to about 35%); and one or more other ingredients (e.g., one or more excipients) (about 15% to about 20%) (percentages correspond to % by weight). In certain of the above embodiments, the one or more plant extracts or other naturally-occurring substances is selected from the group consisting of bauhinia purpurea L. extract, hemerocallis fulva (flower) extract, yohimbe (pausinystalia johimbe) (bark) extract, and higenamine or norcoclaurine. Therefore, in certain embodiments, the composition comprises aegeline, caffeine, bauhinia purpurea L. extract, hemerocallis fulva (flower) extract, yohimbe (pausinystalia johimbe) (bark) extract, and higenamine or norcoclaurine, and in more specific embodiments the composition comprises aegeline (e.g., at about 10% to about 15%); caffeine (e.g., at about 35% to about 45%); bauhinia purpurea L. extract (e.g., at about 15% to about 25%); hemerocallis fulva extract (e.g., at about 0.5% to about 5%); yohimbe (pausinystalia johimbe) (bark) extract (e.g., at about 0.5% to about 5%); and higenamine or norcoclaurine (e.g., at about 5% to about 15%). The compositions of the present invention may further comprise one or more excipients selected from the group consisting of maltodextrin, magnesium stearate, and silicon dioxide. In still further embodiments, the compositions comprise maltodextrin (e.g., at about 1% to about 5%); magnesium stearate (e.g., at about 1% to about 10%); and/or silicon dioxide (e.g., at about 1% to about 10%). While the above embodiments describe the amount of aegeline in terms of percentage of the composition by weight, these percentages may correspond to the dosage and milligram amounts described elsewhere herein. For example, a composition comprising aegeline in an amount of about 40 mg may comprise aegeline in an amount corresponding to about 10% to about 15% of the composition by weight.

Further, while the embodiments described herein primarily concern oral administration of aegeline or of any combination or composition comprising aegeline, the methods and uses of the present invention encompass other routes of administration, including, without limitation, transdermal, intranasal, and other parenteral route.

The administration of aegeline improves mental stamina, focus, energy level, and/or mood, and these effects may accompany a decrease in sense of fatigue. Aegeline administration also increases thermogenesis and affects energy balance, and at relatively higher doses produces anabolic effects and improves physical performance. After repeated or extended administrations, aegeline is ultimately able to affect body composition, including by increasing the percentage of muscle mass and/or decreasing the percentage of fat mass. Aegeline is also able to increase or enhance libido in male mammals. In addition to its unique and unexpected activity profile, aegeline is particularly advantageous because its activities exhibit prolonged duration, which eliminates the need for frequent or multiple administrations to achieve such beneficial effects. Consequently, aegeline's unique properties permit a dosing schedule with fewer administrations compared to a dosing schedule that might be followed when administering compositions that do not comprise aegeline.

### EXAMPLES

The following examples are for illustration only. Persons skilled in the art will appreciate that modifications, to both materials and methods, are within the scope of the invention.

### Example 1

### Aegeline Synthesis

The following synthesis scheme provides an efficient, low cost strategy for producing aegeline. It therefore provides an improved strategy for aegeline synthesis.

The starting material for this synthesis is 1-(4-methoxyphenyl)-ethanone:

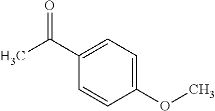

The first step is allylic bromination. Anhydrous ethanol (400 kg) and 1-(4-methoxyphenyl)ethanone (42 kg) were added to a 1000 L vessel and stirred at 25° C. Then N-bromosuccinimide (56 kg) was added potionwise. This mixture was stirred at 40° C. for 3 hours, after which the mixture was cooled to 25° C. Next, hexamethylenetetramine (38 kg) was added potionwise. This mixture was stirred at 25° C. for 3 hours and then filtered. Anhydrous ethanol (560 kg) was added to the precipitated solid in a 1000 L vessel, and the mixture was stirred at 25° C. Concentrated hydrogen chloride (HCl, 35 kg) was then added for acid hydrolysis, and stirring continued at 25° C. for 12 hours. After this time, the volume was concentrated to ⅓, and the temperature was reduced to 5° C. for crystallization. The product was filtered and dried. This product is intermediate 2-amino-1-(4-methoxyphenyl)ethanone hydrochloride (35 kg):

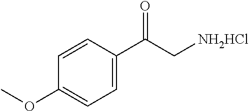

The yield of 2-amino-1-(4-methoxyphenyl)ethanone hydrochloride, based on starting product 1-(4-methoxyphenyl)ethanone, was 62.1%.

Dichloromethane (500 kg, solvent) was added to a 500 L vessel containing the compound 2-amino-1-(4-methoxyphenyl)ethanone hydrochloride (35 kg). Also added was triethylamine (35 kg, organic base; other organic bases include tributylamine, pyridine, tripropylamine, N,N-dimethylaniline and N,N-diethylamine). This mixture was stirred at 10° C., and cinnamoyl chloride (26 kg) was then added dropwise. The mixture was then stirred at 25° C. for 3 hours. Purified water (100 kg) was added dropwise and the mixture stirred at 5° C. to precipitate solid. The mixture was filtered and the resulting cake was washed with the addition of anhydrous ethanol (50 kg). The product was filtered and dried. This product is intermediate N-(2-(4-methoxyphenyl)-2-oxoethyl)cinnamamide (37 kg):

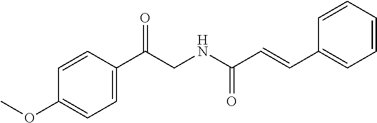 [SMILES: COc1ccc(C(=O)CNC(=O)/C=C/c2ccccc2)cc1]

The yield of N-(2-(4-methoxyphenyl)-2-oxoethyl)cinnamamide, based on starting product 1-(4-methoxyphenyl)ethanone, was 44.8%.

To make the final product aegeline, the ketone was reduced to an alcohol. Anhydrous ethanol (300 kg) was added to N-(2-(4-methoxyphenyl)-2-oxoethyl)cinnamamide (37 kg) in a 500 L vessel. The mixture was stirred at 10° C. and sodium borohydride (6 kg) was added potionwise. Stirring continued at 10° C. for 2 hours, after which the volume was concentrated to ⅓. Purified water (200 kg) was added, and the stirring temperature was reduced to 5° C. The precipitated solid was filtered and then boiled in anhydrous ethanol (500 kg), hot filtered, and cooled. Crystallization proceeded at 5° C., and after filtering the cake was washed in anhydrous ethanol (50 kg). After filtering, the cake was dried. This product, as confirmed by mass spectrometry and H-nuclear magnetic resonance, is aegeline (30 kg). The final yield of aegeline, based on starting product 1-(4-methoxyphenyl)ethanone, was 36.1%.

### Example 2

### Acute Toxicity and Tolerance in Mammals

The maximum tolerated dose (MTD) of orally administered aegeline was determined in New Zealand white rabbits and Wistar rats.

A determination of MTD of orally-administered aegeline in New Zealand white rabbits was carried out as follows. Twenty-four rabbits (twelve male, twelve female) were quarantined for seven days and then were randomly divided into four groups with six animals (3 male and 3 female) in each group. The groups were treated as follows: Group I (vehicle control—dimethyl sulfoxide (DMSO)), Group II (500 mg/kg), Group III (1000 mg/kg), and Group IV (2000 mg/kg). Average weight of the animals was 1.5-2.0 kg. Aegeline was administered orally through a stomach feeding tube.

Animals were observed daily for fourteen days following administration. Each animal's body weight, feed intake, mortality, and natural orifices were monitored. Animals were also observed for clinical signs at 15 minutes, 30 minutes, 1 hour, 2 hours, 4 hours, and 6 hours post administration on the day of administration, and once daily for each subsequent day of the study. Clinical observations included tremor, convulsion, piloerection, salivation, lacrimation, respiration, activity, arched back, distended abdomen, skin condition, fur, mucous membrane, presence or absence of secretions, eye condition, tail elevation, motor activity, posture, gait, urination and defecation, neurological disorders, presence of clonic and tonic movements.

All animals were observed daily for any overt toxicity. Regular observations included observation of natural orifices for any abnormal secretions, skin condition, mortality, gait, movement, neurological disorders, eye condition, feces, convulsions, tremors, activity rate, restlessness, grooming, sleep, aggressiveness, salivation, diarrhea, respiration, appetite, thirst and urine conditions. Other observations such as fur condition, mucous membranes, presence or absence of secretions and excretions and autonomic activity, lacrimation, piloerection, pupil size, posture and response to handling as well as the presence of clonic or tonic movements, repetitive circling, bizarre behavior, self-mutilation and walking backwards were also recorded. Body weight of each animal was recorded on the day of dosing and once daily for 14 days during the experimental period.

Necropsy and gross pathological examination was carried out on all animals after the test period. Heart, lung, liver, kidneys, spleen, stomach, large intestine, small intestine, brain, skin, thymus, adrenal glands, duodenum, jejunum, caecum, colon and rectum were examined for any macroscopic lesions.

The animals were found to be normal during entire study period and no clinical signs were observed in any animal treated with vehicle control or test article (aegeline) at the doses of 500, 1000 and 2000 mg/kg body weight. No pre-terminal mortality was recorded during the entire study period. The body weight gain and feed intake of all the animals in vehicle control and treatment groups were normal. Gross pathology examination revealed no abnormal changes in the vehicle control or treatment group animals. The MTD of a single oral dose of aegeline was determined to be greater than 2000 mg/kg in New Zealand white rabbits.

The same general methods and procedures were followed as described above to determine MTD of orally-administered aegeline in Wistar rats. This study involved forty animals randomly divided into four groups of ten animals (five male, five female) in each group. Animals were 7-8 weeks old with average weight between 180-200 grams. The animals were administered aegeline or vehicle control by oral gavage as follows: Group I (vehicle control—dimethyl sulfoxide (DMSO)), Group II (500 mg/kg), Group III (1000 mg/kg), and Group IV (2000 mg/kg).

The animals were found to be normal during the entire study period and no clinical signs were observed in any animal treated with vehicle control or test article at the different doses of 500, 1000 and 2000 mg/kg body weight. No pre-terminal mortality was recorded during the entire study period. The body weight gain and feed intake of all animals in the vehicle control and treatment groups were normal. Gross pathology examination revealed no abnormal changes in any animal from the vehicle control or treatment groups. The MTD of a single oral dose of aegeline was determined to be greater than 2000 mg/kg in Wistar rats.

### Example 3

### Sub-Chronic Toxicity in Animals

Ninety-day, repeated dose toxicity is determined in New Zealand white rabbits and Wistar rats.

To determine sub-chronic toxicity in New Zealand white rabbits, forty-eight New Zealand white rabbits (24 male, 24 female) will be randomly divided into six groups consisting of 8 animals (4 male, 4 female) in each group, with treatments as follows:

TABLE 1
Repeat dose study in New Zealand white rabbits

| Group No. | Group description | Dose (mg/kg) |
|---|---|---|
| I | Vehicle control | 0 |
| II | Low dose | 25 |
| III | Mid dose | 50 |
| IV | High dose | 100 |
| V | Vehicle control - recovery | 0 |
| VI | High dose - recovery | 100 |

The animals will receive either aegeline (in 20% DMSO) or vehicle control (20% DMSO in water) by oral gavage daily for 90 days. Animals in groups I-IV are observed for 90 days after the start of treatment, while animals in groups V and VI are observed for an additional 14 days (the recovery period) after the treatment period, for a total of 104 days of observation following the start of treatment. The animals will be observed once daily for clinical signs for a period of 90 days for the main and recovery groups animals, and for an additional 14 days after the last dosing day for the recovery group animals. Mortality will be observed twice daily, once in the morning and once in the evening, for 90 days for the main and recovery groups animals, and for an additional 14 days after the last dosing day for the recovery group animals.

All animals will be observed daily for any general behavioral and physical changes. The regular observation will include natural orifices, skin condition, mortality, gait, movement, neurological disorders, eye condition, feces, and urine conditions. These observations will be made daily once outside the home cage. Other observations including skin, fur, eyes, mucous membranes, presence or absence of secretions and excretions and autonomic activity, lacrimation, piloerection, pupil size, unusual respiratory pattern, changes in gait, and posture will also be recorded. Body weight and feed intake of each animal will be recorded once each week during the study period. Toxicity will be assessed in terms of general behavior, mortality, serum biochemistry, hematology, urine analysis and histopathology examinations.

A functional observation battery (FOB) test will be conducted during the last week of the experimental period and will include observations such as home-cage measurement, hand-held observation, open field activity, stimulus response, nervous and muscle measurement, body temperature, locomotor activity, and urine analysis. Ophthalmologic examination will be carried out at the end of the treatment period on all control and high dose animals. The examinations may be extended to lower doses if any apparent changes are noticed in the high dose groups. On the last day of dose administration, all rabbits of main groups will be kept in metabolic cages for approximately 12 hours for urine collection. The urine will be collected in tarson tubes individually and subjected to analysis for specific gravity, pH, protein, glucose, blood/blood cells along with appearance, and volume. The recovery group animals will be subjected to urine collection fourteen days after the last day of dose administration for analysis by the same way as main group animals.

On the last day of dose administration, all rabbits will be kept for overnight fasting, with only free access to water. On the day after the 90-day dosing period, blood samples will be collected from each rabbit of the main groups through marginal ear vein in vials containing anticoagulant (4% K2 EDTA at 0.03 mL/1 mL of blood), and the samples will be used for hematological analysis. For clinical chemistry parameters, blood samples will be collected in vials and after clotting the samples will be centrifuged and serum will be collected. For the recovery groups animals, the animals will be kept for overnight fasting on the last day of the recovery period, with only free access to water, and the next day the animals will be subjected to blood sampling for hematology and biochemistry analyses as described for the main groups. Hematology parameters will include hemoglobin, total RBC count, hematocrit, total WBC count, platelet count, differential leukocyte count (eosinophil, basophil, neutrophil, lymphocyte, monocyte). Clinical Chemistry parameters will include glucose, blood urea nitrogen (BUN), creatinine, triglycerides, cholesterol, total protein, albumin, globulin (calculated), billirubin, aspartate aminotransferase (AST), alanine aminotransferase (ALT), alkaline phosphatase (ALP), sodium, potassium, phosphorous, and calcium.

After blood sample collection, animals will be sacrificed by intravenous overdose of barbiturate and necropsy will be performed. All animals that die during the study period, as well as all surviving animals in the study, shall be subjected to a full, detailed gross necropsy, which includes careful examination of the external surface of the body, all orifices, and the cranial, thoracic and abdominal cavities and their contents.

After necropsy (macroscopic observation), organs such as lungs, liver, heart, kidneys, spleen, brain, epididymides, thymus, and testes/uterus with ovaries will be dissected free of fat and weighed wet as soon as possible to avoid drying. The absolute organ weights will be used to estimate the organ-body weight ratio (relative) by using the fasting body weights. Organs such as brain, thyroid, spleen, epididymides, thymus, adrenals, lung, heart, skin, esophagus, stomach, duodenum, jejunum, terminal ileum, colon, liver, kidneys, testes/uterus and ovaries will be collected in containers with 10% formalin solution for histopathology.

All tissues will be processed and stained with hematoxylin and eosin. Histopathological examination will be restricted to the specified list of tissues, including all macroscopically abnormal tissues from all animals from the control (G1) and high dose (G4) groups sacrificed at termination, and also from the animals from the control recovery (G5) and high dose recovery (G6) groups sacrificed after the recovery period. A 5-step grading system of minimum, mild, moderate, marked, and severe will be used to rank microscopic findings for comparison among groups. If there are any specific organs showing treatment-related changes in the high dose group, histopathological examination of those organs will be extended to all other dosage groups.

Sub-chronic toxicity in Wistar rats was determined as follows. After a quarantine period, 100 healthy animals (50 male, 50 female; body weight 100-120 g; aged 5-7 weeks), were randomized and divided into the following groups:

TABLE 2
Repeat dose study in Wistar rats

| Group No. | Number of animals | Group description | Dose (mg/kg) |
|---|---|---|---|
| I | 10 male, 10 female | Vehicle control | 0 |
| II | 10 male, 10 female | Low dose | 50 |
| III | 10 male, 10 female | Mid dose | 100 |
| IV | 10 male, 10 female | High dose | 200 |
| V | 5 male, 5 female | Vehicle control - | 0 |
| | | recovery | |
| VI | 5 male, 5 female | High dose - recovery | 200 |

The body weight variation among the groups did not exceed ±20%. The test article (in 40% DMSO) and vehicle (40% DMSO) was administered once daily by oral gavage for 90 days. The animals were observed daily for clinical signs for a period of 90 days, and animals in Groups V and VI were observed for an additional 14 days (or for 104 days from the day of starting dosing).

Observations included natural orifices, skin condition, mortality, gait, movement, neurological disorders, eye condition, feces and urine conditions, convulsions, tremors, activity rate, restlessness, grooming, sleep, aggressiveness, salivation, diarrhea, appetite, thirst, as well as fur, eyes, mucous membranes, presence or absence of secretions and excretions and autonomic activity, lacrimation, piloerection, pupil size, unusual respiratory pattern, changes in gait, posture, response to handling, presence of clonic or tonic movements, repetitive circling, bizarre behavior, self-mutilation, and walking backwards. Body weight and feed intake of each animal was recorded once per week during the study period (body weight was recorded beginning on day 1; feed intake was recorded beginning on day 8). Mortality was observed daily.

FOB test was conducted at the end of the 11th week of exposure to assess the impact of aegeline on neuromotor activity. Sensory reactivity to graded stimuli of different types (auditory, visual, and proprioceptive stimuli), motor reactivity, grip strength, and rotarod activity (run time per second) were observed.

The grip strength test measures the effect of a test article on the neuro-muscular system by determining the limb strength of rodents. Using a Grip Strength Meter (Chatillon, Columbus), each animal was placed on the top of the grid such that only the fore limbs could initially grip the grid, keeping the torso horizontal. The animal was then pulled by its tail gently without jerking until the grip was completely released down the complete length of the grid. The maximal grip strength at which the animal releases the grid was recorded in Gram Force (GF). The same procedure was repeated by placing the animal on the grid such that both fore limbs as well as hind limbs could grip the grid. The grid was cleaned with 70% ethanol and dried with tissue before testing each animal.

A rotarod performance test was conducted to evaluate balance and coordination of rats under the influence of aegeline. The test involves a rotating rod that is suspended at a height that induces avoidance of a fall and that is rotated at a specified speed. The rod is divided into channels or lanes, and a rodent is placed on the rotating rod in one of the channels. In this study, animals were placed on the channels of a Rotamex-5 025402002L (Columbus), with an empty channel in between channels with animals. The animals walked forward to maintain their balance and positioning at an initial speed of 4 RPM for 60 seconds. The apparatus was then accelerated from 4 to 40 RPM in 300 seconds. As an animal lost its grip and fell, the speed of the rod at the time of fall, as well as the amount of time the animal stayed on the rod, were automatically recorded. The surface of the rod, the side walls and the floor under the rod were cleaned with 70% alcohol before wiping the apparatus dry for testing the next set of rats.

Ophthalmologic examination was performed on control and treated animals prior to the initiation of dosing and in the 13th week (for Groups I-IV) and 15th week (for Groups V and VI) of the study. Eye examination was carried out using a hand slit lamp after induction of mydriasis with 1% solution of cyclopentolate hydrochloride.

For urinalysis, animals were kept overnight in metabolic cages on the last day of the experiment for urine collection. Samples were collected and analyzed individually using a urine analyzer (DIRUI 100 Urine Analyzer) for the following parameters: visual, color, specific gravity, pH, protein, glucose, ketone bodies, urobilinogen, nitrites, and blood.

On the last day of the study, rats were kept for overnight fasting with only free access to water. On the following day (day 91 for Groups I-IV and day 105 for Groups V and VI), blood samples were collected through retro-orbital plexus in two vials—one vial containing anticoagulant (4% EDTA at 0.03 mL/1 mL of blood) for hematological analysis, and the other vial devoid of anticoagulant to collect serum for biochemical analysis. The hematology parameters were analyzed using a Seimen's Advia 120; these parameters included hemoglobin, total erythrocyte count, hematocrit, total leucocyte count, platelet count, granulocytes, monocytes, and lymphocytes. The clinical chemistry parameters were analyzed using a Seimen's Dimension Expand Plus; the parameters measured were glucose, blood urea nitrogen, creatinine, triglycerides, cholesterol, total protein, total bilirubin, aspartate aminotransferase, alanine aminotransferase, alkaline phosphatase, sodium, potassium, phosphorous, and calcium.

After blood sample collection, the animals were sacrificed using the carbon dioxide asphyxiation method and necropsy (macroscopic observation) was performed. Vital organs were collected and weighed and were further processed for histology.

The data for body weights, feed intake, hematology, serum biochemistry and organ weights were analyzed as follows: ANOVA test (R-code program) was applied to test for homogeneity of variance between groups. If significant, heterogeneity of variance was determined using the Tukey multiple comparison test. Variations were considered significant at p<0.05.

For histopathology, brain, thyroid, spleen, adrenal glands, lung, heart, skin, stomach, duodenum, jejunum, terminal ileum, colon, liver, kidneys, testes/uterus and ovaries were examined for any gross lesions. The organs were collected and fixed in 10% neutral buffered formalin solution. The organs were processed using routine methods for embedding in paraffin, sectioning, and staining with hematoxylin and eosin.

Results: In the present study, all animals treated with repeated oral doses of aegeline for 90 days survived through the entire study period; no pre-terminal mortality was recorded. Further, no changes were observed in the behavior during cage-side observations. A normal body weight gain was observed in both males and females across all groups during the entire study period. Feed intake was also found to be normal during the entire study period across all groups. The functional observation battery (FOB) test revealed no abnormalities. Similarly, the rotarod and grip strength experiments did not reveal any dose-related effect: in the grip strength experiment, no significant difference was observed (for fore limbs separately, as well as fore limbs and hind limbs together) between the control and treatment groups for both the sexes; and the mean run time in the rotarod experiment did not reveal any dose-dependent changes in treatment groups as compared to the control groups in males and females. Opthalmoscopic examination also did not reveal any abnormalities.

There were no treatment-related changes in hematology, clinical chemistry, urinalysis, or absolute and relative organ weight parameters, for both male and female animals. With respect to the hematology analysis, all of the parameters were within the normal range across all groups. All of the parameters in serum biochemistry analysis were within the normal range in the treatment and control groups; there were no treatment-related effects on clinical chemistry parameters. In addition, there was no significant difference in mean absolute organ weights in treatment groups as compared to control group in both male and female animals, and there was no significant difference in mean relative organ weights in treatment groups as compared to control group in both male and female animals. In general observations, no behavioral abnormalities were observed in any of the experimental animals during the study period.

During gross pathology examinations, no treatment-related abnormalities were observed. Some observations, such as multiple red foci in lungs, uterine hydrometra, hepatic cyst, focal nodular swelling and multiple white foci in the liver, were recorded in control and treatment animals and thus were not associated with aegeline treatment.

Histopathology observations indicated minimal to moderate aberrations in the liver, lungs, brain, kidneys, spleen, stomach, prostate gland, thymus and uterus. But these changes were observed in both control and high-dose group animals. The changes in lung were detected as diffused alveolar congestion, focal alveolar histiocytosis, perivascular multifocal mononuclear cell (MNC) infiltration, diffused alveolar emphysema, multifocal thickening of alveolar wall, focal alveolar MNC infiltration, diffuse alveolar oedema, multifocal alveolar emphysema, and presence of keratin cast in alveoli (focal). The kidneys showed mineralization of renal papilla (focal), peritubular focal MNC infiltration in cortex and medulla, renal tubular degeneration, focal chronic inflammation of cortex, perivascular multifocal MNC infiltration, focal basophilic renal tubules (cortex), and chronic inflammation of pelvic subepithelium. The changes in liver included focal and multifocal microgranulomma, focal MNC infiltration, multifocal chronic inflammation, focal hepatocyte necrosis accompanied with MNC infiltration, single cell necrosis of hepatocyte (focal), perivascular and periportel MNC infiltration. Brain histological changes were recorded as focal MNC infiltration in cerebrum (cortex), focal perivascular cuffing in cerebellum. The spleen histopathological abnormalities were observed as increased extramedullary hematopoiesis (EMH) (diffused), focal and multifocal increased EMH, and congested red pulp. For prostate, one male animal in the vehicle control group showed peritubular focal MNC infiltration. A female animal in the vehicle control group showed histological changes in the stomach, which were recorded as focal MNC infiltration in glandular mucosa and submucosa. In uterus, changes were dilated lumen and thinning of uterine wall. One male animal in the high dose group exhibited minimal aberration in the thymus, which was recorded as single cell diffused necrosis of thymic lymphocytes (cortex). In general, changes were observed in a few animals across treatment and control groups, and hence changes may be attributed to background pathology rather than to test treatment.

Oral administration of aegeline once daily for 90 days did not cause any adverse effect in Wistar rats at the dose rates of 50.0 mg/kg, 100.0 mg/kg and 200.0 mg/kg body weight under laboratory conditions. The results obtained from the acute Maximum Tolerated Dose (MTD) study showed that MTD of aegeline administered orally was greater than 2000 mg/kg body weight in rats, and the doses used in this sub-chronic study suggest that daily doses of aegeline greater than 1/10 the MTD can be well-tolerated.

### Example 4

### Effects of Aegeline in Humans

The effects of aegeline administration were tested in human subjects. In an initial study, two male subjects ingested 20-150 mg of aegeline for 12-18 weeks. At doses of 20-60 mg per day, the individuals reported no immediate apparent effects. However, at doses of 80-150 mg per day, the subjects reported that aegeline ingestion increased or improved mental stamina, focus and concentration, energy level, and mood. Specifically, a daily dose of 80 mg aegeline was effective to increase or improve mental stamina, focus, and mood, without a very noticeable effect on thermogenesis. A daily dose of 120 mg aegeline further increased or improved mental stamina, focus, and mood; this daily dose of 120 mg aegeline produced these effects to a desirable degree and for a desirable duration (e.g., about 8-10 hours after effect onset). In addition, a daily dose of 120 mg aegeline produced a noticeable but not very apparent thermogenic effect. An additional dose increase to 150-180 mg further enhanced or increased all of these effects (i.e., both mental stamina, focus, energy level, and mood, as well as thermogenesis). In sum, this initial study demonstrated that (i) the cognitive and thermogenic effects resulting from aegeline ingestion are dose-dependent, and (ii) aegeline can improve mental stamina, focus, energy level, and/or mood at a lower dose compared to the dose threshold effective to increase thermogenesis, such that aegeline can be administered in an amount effective to increase or improve mental stamina, focus, energy level, and/or mood without increasing thermogenesis.

The two male volunteers also reported that aegeline ingestion increased libido. This effect was observed at a daily dose of 150 mg aegeline but not at the lower daily doses. Accordingly, aegeline may be administered in an amount that can improve mental stamina, focus, energy level, and/or mood without increasing libido. Alternatively, aegeline may be administered in an amount that improves mental stamina, focus, energy level, and/or mood, as well as libido.

In a separate study, the compound aegeline was orally administered to six human subjects. The volunteer subjects were all non-smoking males between the ages of 24-36. The subjects were instructed to cease using any other dietary supplements for two weeks prior to the start of aegeline ingestion, but to otherwise continue typical diet intake and exercise schedule (which was 4-6 times per week of exercise). All subjects ingested 200 mg of aegeline per day (capsule form, with or without food) for 11-16 days. For two of the subjects, aegeline ingestion was increased to 400 mg for at least 4 days. One of these subjects further increased his daily dose to 600 mg for 5 additional days, and then increased the daily dose again to 800 mg for 6 days thereafter.

Blood samples were collected from each subject on the first day of the study (Day 0), prior to the first aegeline serving (baseline on Day 0), and after 2-5, 6-9, and 11-16 days of aegeline ingestion, with the actual days of sampling depending on the subject. Because the baseline day and first day of dosing is Day 0, a blood draw on Day 3, for example, reflects effects resulting from 3 days of aegeline in the system. From the two subjects extending aegeline intake past Day 16, blood samples were also collected on Days 21-23. Blood samples were collected from one of these subjects on Days 28 and 34, following an ingestion escalation to 600 and 800 mg/day, respectively. All samples were submitted to a commercial diagnostics laboratory (Laboratory Corporation of America, Dublin, Ohio and Burlington, N.C.) for analysis, including a complete metabolic panel (14 endpoints), sex hormone levels (6 endpoints), and thyroid function indicators (4 endpoints). The endpoints analyzed were selected to determine if aegeline consumption at 200 mg/day would result in changes (outside of standard reference levels) in blood indicators of liver, kidney, and thyroid function, electrolyte balance, and sex hormone levels.

TABLE 3
Summary of human study protocol of
six adult males ingesting 200-800 mg aegeline per day

| | | Daily dose | | |
|---|---|---|---|---|
| Subject | | ingested | | |
| No. | Age | (mg)* | Days ingested | Days blood drawn** |
| 1 | 35 | 200 | Days 0-12 | Days 0, 2, 6, and 13^{♦} |
| | | 400 | Days 13-22 | Day 23^{♦} |
| | | 600 | Days 23-27 | Day 28^{♦} |
| | | 800 | Days 28-34 | Day 34 |
| 2 | 25 | 200 | Days 0-15 | Days 0, 4, 8, and 15 |
| 3 | 33 | 200 | Days 0-17 | Days 0, 3, 12, and 18^{♦} |
| | | 400 | Days 18-22 | Day 23^{♦} |
| 4 | 33 | 200 | Days 0-14 | Days 0, 2, 7, and 14 |
| 5 | 33 | 200 | Days 0-12 | Days 0, 4, 8, and 12 |
| 6 | 28 | 200 | Days 0-11 | Days 0, 3, 7, and 11 |

*Subjects were instructed to take the daily dose at the same time each day. For days of blood draw (with the exception of Day 0), subjects were instructed to take the daily dose one hour before blood draw, even if this time differed from the normal daily dosing time.
**On Day 0, blood was drawn pre-ingestion to determine each subject's baseline.
^{♦}For Subject Nos. 1 and 3, blood was drawn on the same days of dose escalation. On these days of dose escalation and blood draw, the subjects ingested the previous daily dose prior to blood draw, and then escalated the dose that same day after blood was drawn. For example, on Day 13, Subject No. 1 ingested 200 mg aegeline prior to blood draw, and after blood draw ingested another 200 mg to escalate the daily dose to 400 mg total. Thus the daily dose on Day 13 for Subject No. 1 was 400 mg, while the blood draw on Day 13 corresponded to a daily dose of 200 mg prior to dose escalation.

The only changes from baseline in indicators observed were slight increases in blood urea nitrogen (BUN) levels above reference ranges in two of the six subjects, and a reduction of thyroid stimulating hormone (TSH) in one subject (see Table 4). In two subjects, BUN levels increased from baseline value of 19-20 mg/dL (the upper limit of the reference range) to 22-23 mg/dL at 2-14 days, but BUN levels returned to 18-19 mg/dL by the final blood draw. While BUN levels were slightly elevated, creatinine levels and estimated glomerular filtration rate were unchanged in both of these subjects. These levels may result from high protein diets, slight dehydration at the time of sampling, or both. The subject exhibiting lower-than-baseline TSH values at days 3-11 also had free T3 (triiodothyronine) level (5.2 ng/dL) above the reference range (2.0-4.4 ng/dL) prior to aegeline intake, indicating higher thyroid hormone levels in blood not associated with aegeline intake.

Other than the slight increases in BUN levels in two of six subjects and lower TSH levels in one subject, no other changes from baseline of all other endpoints were seen in the other subjects throughout the observation period. The higher BUN and lower TSH levels in three of the subjects were not related to aegeline intake. Thus, according to the blood analyses, daily intake of 200 mg aegeline for approximately two weeks in six adult males (and up to 800 mg/day in one male) did not result in adverse effects.

TABLE 4
Results (mean ± standard deviation) of complete metabolic panel, sex
hormone, and thyroid function blood tests from six adult males ingesting ≧200 mg
aegeline per day

| | Reference | | Days | Days | Days | Days |
|---|---|---|---|---|---|---|
| Parameter | Range | Baseline | 2-5 | 6-9 | 11-16 | 21-23^{a} |

Metabolic Panel

| | | | | | | |
|---|---|---|---|---|---|---|
| Glucose | 65-99 | 86.8 ± 12.8 | 91.8 ± 4.3 | 88.7 ± 9.1 | 90.0 ± 7.6 | 92.0 ± 9.0 |
| (mg/dL) | | | | | | |
| Blood urea | 6-20 | 21.2 ± 4.5 | 20.5 ± 4.3 | 21.0 ± 6.6 | 19.7 ± 4.9 | 18.5 ± 0.5 |
| nitrogen | | | | | | |
| (mg/dL) | | | | | | |
| Creatinine | 0.76-1.27 | 1.1 ± 0 | 1.0 ± 0.1 | 1.1 ± 0.1 | 1.1 ± 0.1 | 1.0 ± 0.0 |
| (mg/dL) | | | | | | |
| Estimated | >59 | 93.3 ± 8.6 | 98.3 ± 10.2 | 97.0 ± 10.1 | 95.8 ± 7.3 | 102 ± 12 |
| GFR (ml/min/ | | | | | | |
| 1.73) | | | | | | |
| BUN/creatinine | 8-19 | 19.5 ± 3.8 | 19.5 ± 3.7 | 19.7 ± 5.6 | 18.3 ± 4.1 | 18.0 ± 0.0 |
| ratio | | | | | | |
| Sodium | 134-144 | 139.2 ± 0.9 | 139.2 ± 1.2 | 139.0 ± 1.3 | 139.7 ± 0.9 | 139.5 ± 0.5 |
| (mmol/L) | | | | | | |
| Potassium | 3.5-5.2 | 4.3 ± 0.1 | 4.2 ± 0.4 | 4.2 ± 0.2 | 4.2 ± 0.3 | 4.5 ± 0.0 |
| (mmol/L) | | | | | | |
| Chloride | 97-108 | 101.3 ± 1.6 | 102.0 ± 2.0 | 101.2 ± 1.2 | 101.3 ± 1.2 | 103.0 ± 1.0 |
| (mmol/L) | | | | | | |
| Total carbon | 20-32 | 23.5 ± 1.7 | 23.2 ± 1.8 | 23.5 ± 1.5 | 23.8 ± 1.2 | 22.0 ± 1.0 |
| dioxide | | | | | | |
| (mmol/L) | | | | | | |
| Calcium | 8.7-10.2 | 9.7 ± 0.3 | 9.6 ± 0.2 | 9.7 ± 0.2 | 9.6 ± 0.3 | 9.5 ± 0.3 |
| (mg/dL) | | | | | | |
| Protein - total | 6.0-8.5 | 7.1 ± 0.4 | 7.1 ± 0.4 | 7.2 ± 0.2 | 7.1 ± 0.3 | 6.9 ± 0.0 |
| (g/dL) | | | | | | |
| Albumin | 3.5-5.5 | 4.6 ± 0.2 | 4.6 ± 0.2 | 4.7 ± 0.1 | 4.6 ± 0.1 | 4.5 ± 0.1 |
| (g/dL) | | | | | | |
| Globulin - | 1.5-4.5 | 2.4 ± 0.3 | 2.5 ± 0.4 | 2.5 ± 0.3 | 2.5 ± 0.3 | 2.4 ± 0.1 |
| total (g/dL) | | | | | | |
| Albumin/globulin | 1.1-2.5 | 1.9 ± 0.3 | 1.9 ± 0.4 | 1.9 ± 0.3 | 1.9 ± 0.3 | 1.9 ± 0.1 |
| ratio | | | | | | |
| Bilirubin - | 0-1.2 | 0.8 ± 0.4 | 0.7 ± 0.3 | 0.8 ± 0.3 | 0.7 ± 0.4 | 0.8 ± 0.4 |
| total (mg/dL) | | | | | | |
| Alkaline | 25-150 | 74.7 ± 20.6 | 74.7 ± 20.7 | 76.2 ± 22.2 | 76.7 ± 21.3 | 89.0 ± 10.0 |
| phosphatase | | | | | | |
| (IU/L) | | | | | | |
| AST (SGOT) | 0-40 | 28.8 ± 7.9 | 26.3 ± 5.4 | 27.7 ± 6.9 | 26.2 ± 4.7 | 24.5 ± 0.5 |
| (IU/L) | | | | | | |
| ALT (SGPT) | 0-55 | 33.2 ± 17.8 | 32.0 ± 17.4 | 30.8 ± 17.4 | 31.1 ± 17.4 | 21.0 ± 2.0 |
| (IU/L) | | | | | | |

Sex Hormone

| | | | | | | |
|---|---|---|---|---|---|---|
| Testosterone - | 348-1197 | 446 ± 228 | 499 ± 234 | 493 ± 260 | 486 ± 221 | 705 ± 198 |
| total | | | | | | |
| (ng/dL) | | | | | | |
| Testosterone - | 8.7-25.1 | 10.4 ± 4.4 | 9.9 ± 3.7 | 10.2 ± 4.7 | 11.6 ± 4.2 | 12.5 ± 1.3 |
| free (pg/dL) | | | | | | |
| LH (mIU/mL) | 1.7-8.6 | 4.3 ± 1.8 | 3.6 ± 1.0 | 5.1 ± 2.4 | 4.7 ± 1.9 | 7.0 ± 0.2 |
| FSH | 1.5-12.4 | 4.6 ± 3.5 | 4.4 ± 3.4 | 4.8 ± 4.1 | 4.9 ± 4.2 | 8.3 ± 5.8 |
| (mIU/mL) | | | | | | |
| Estradiol | 7.6-42.6 | 26.2 ± 10.2 | 22.5 ± 9.9 | 22.7 ± 13.1 | 22.5 ± 110.7.4 | 33.0 ± 12.8 |
| (pg/mL) | | | | | | |

Thyroid Function

| | | | | | | |
|---|---|---|---|---|---|---|
| Thyroxine | 4.5-12.0 | 8.0 ± 2.3 | 8.0 ± 1.9 | 8.7 ± 2.0 | 8.4 ± 1.9 | 8.3 ± 0.3 |
| (T4) (μg/dL) | | | | | | |
| Triiodothyronine | 71-180 | 105 ± 23 | 104 ± 22 | 108 ± 29 | 108 ± 26 | 89 ± 2 |
| (T3) | | | | | | |
| (ng/dL) | | | | | | |
| Triiodothyronine | 2.0-4.4 | 3.6 ± 0.9 | 3.4 ± 0.7 | 3.5 ± 1.0 | 3.5 ± 0.9 | 3.0 ± 0.1 |
| (T3) - free | | | | | | |
| (pg/dL) | | | | | | |
| TSH (μIU/mL) | 0.45-4.5 | 1.7 ± 1.1 | 2.0 ± 1.3 | 1.9 ± 1.3 | 1.9 ± 1.1 | 1.4 ± 0.1 |
| Sex hormone | 16.5-55.9 | 29.3 ± 15.1 | 31.8 ± 16.6 | 33.0 ± 17.7 | 31.7 ± 16.3 | 41.1 ± 13.3 |
| binding | | | | | | |
| globulin | | | | | | |

^{a}Two subjects increased the daily intake to 400 mg aegeline/day prior to Day 21.

In addition to the blood sampling data discussed above, the subjects reported the following effects after taking varying doses of aegeline:

Cognitive effects: Aegeline's effects on mental stamina, energy level, focus, and mood were reported to be very apparent at a daily dose of 200 mg. These effects were further increased or improved when compared to the lower daily doses of 80-180 mg. However, the dose-dependency of these effects was not readily observed at doses above 400 mg aegeline per day. Accordingly, a daily dose above ˜400 mg aegeline is not required to experience the maximum benefits of aegeline ingestion on mental stamina, energy level, focus, and mood.

Thermogenesis: As discussed above, aegeline's thermogenic effect was not very noticeable at the 80 mg daily dose but is noticeable at the 120 daily dose. At 200 mg aegeline per day, the subjects reported that aegeline's thermogenic effect was very apparent. The subjects reported feeling an increase in body temperature, such as feeling warm and hot.

Libido: Also as discussed above, a daily dose of 150 mg aegeline was effective to increase libido in the male volunteers. The 200 mg/day study demonstrated that aegeline's effect on libido is further increased at a daily dose of 200 mg, and that an additional increase in dose to 400 mg per day results in a very apparent and prolonged increase in libido. The subjects taking 400 mg aegeline per day reported high libido and a relatively persistent increase in libido.

Anabolic effects: Aegeline can also produce anabolic effects, although the dose threshold for these effects is higher than the dose thresholds for the other effects discussed above. For example, the subjects reported that a daily dose of 200 mg aegeline per day resulted in anabolic effects that became apparent after extended use of aegeline (e.g., at around 7 days of daily administration). However, increasing the daily dose to 400 mg produced anabolic effects that were immediately apparent. Such anabolic effects included noticeable changes in body composition, such as increased muscle fullness and density and reduced body fat, as well as more intense muscle contraction during isolation exercises. The subject who increased his daily dose to 800 mg also reported improved physical performance for exercise and strength training activities during the time of administration. The improved physical performance included the ability to endure higher intensity exercises, and to require less warm-up to begin exercise training.

In addition to the aforementioned studies, two male volunteers ingested a combination of aegeline (60-1000 mg) and caffeine (200-300 mg) for 6-8 weeks. The subjects reported that a combination of 80-150 mg of aegeline and 200-300 mg caffeine increased mental stamina, energy level, focus, and mood, and further that the addition of aegeline to caffeine eliminated the crash effect typically experienced with caffeine ingestion. The subjects reported that a combination of >150 mg aegeline and 200-300 mg caffeine also increased mental stamina, energy level, focus, and mood, but also produced a very apparent anabolic effect (described as more intense muscle contractions and denser muscles). The anabolic effects became much more apparent with continued use of this combination.

Further observations of the effects of aegeline were recorded following a 30-day period of daily administrations of 400 mg aegeline. In this evaluation, normal, healthy male volunteers were instructed to self-administer one capsule daily for 30 days. The identity of the contents and ingredients of the capsule were not disclosed to the volunteers. They were instructed that the test agent should result in one or more effects (though the type and nature of effect(s) was not disclosed) and were asked to complete questionnaires to record any such effects. The volunteers completed the questionnaire before taking the first dose and again on the final day (day 30) of the study period. The questionnaires listed twenty-seven variables and asked each volunteer to subjectively rate the variables on a visual analog scale (VAS; see below for description of VAS). The questionnaire listed the following variables: increased alertness; decreased appetite; bad dreams/nightmares; fatigue; increased energy; dizziness; fainting; rapid heart-beat; cold sweats; improved workouts; restlessness and anxiety; weakness; rapid breathing; improved mental focus; higher risk of infection (frequent illness); nausea with vomiting; increased strength; difficulty sleeping; headache; mood swings or other emotional changes; improved physique appearance; restlessness; dry mouth/increased thirst; stomach upset; improved mental outlook and mood; chest pains; depression. The questionnaire asked each volunteer to indicate, for each variable, “Yes or No” as to whether he experienced the variable at any time during the past two weeks, and if yes, to further indicate “Mild, Moderate, Severe.” A group of 31 volunteers completed and returned questionnaires online for both baseline prior to dosing and end of study. Volunteers were able to interact with each other and share thoughts about the effects they experienced. A paired t-test (two-tailed) was used to evaluate the changes reported from baseline to study completion, with statistical significance set at p<0.01, for the following variables: increased alertness, increased energy, improved workouts, improved mental focus, increased strength, improved physique appearance, improved mental outlook and mood, and dry mouth. All of these variables, with the exception of dry mouth, achieved statistical significance. While no statistically significant change was reported for dry mouth, changes reported for improved physique appearance were significant with p=0.00102, and changes for the other variables were significant with p-values <0.001.

### Example 5

### Comparison Studies

This prophetic study involves a comparison of single-dose administrations of aegeline and caffeine, administered alone or in combination, to humans in order to determine the duration and nature of the acute effects of each compound alone and in combination on mental stamina, energy level, focus, and mood, as well as on thermogenesis, libido, and anabolic effects. In a double-blinded cross-over test, six to eight healthy, non-smoking subjects of normal weight are randomly assigned to receive aegeline or caffeine or a combination of both substances. Separate test sessions are conducted, such that all of the subjects receive each of the different treatments but on different days, thereby generating multiple data points for each treatment group. Doses/treatments will be separated by at least three days. The subjects are instructed to refrain from exercise during the 24 hours prior to the morning of administration, and no caffeine or stimulatory dietary supplements or performance drinks should be used during the 24 hours before and after dosing.

On the morning of administration or ingestion, and following an overnight fast, the subjects arrive to a testing room, are weighed, and then are asked to sit quietly for ten minutes. Following this quiet rest period, each subject's heart rate (HR), systolic blood pressure (SBP), and diastolic blood pressure (DBP) are measured and recorded as a baseline. Each subject is asked to wait ten additional minutes, after which heart rate and blood pressure are measured a second time for the baseline. Each subject is then given a test substance, which is either aegeline, caffeine, or a combination of both aegeline and caffeine. Aegeline is administered in an amount of 60-1000 mg and caffeine is administered in an amount of 250-300 mg or about 3-4 mg/kg. Other doses or amounts can alternatively or additionally be tested. Each subject ingests his test substance and is instructed to sit quietly in the test room. Each subject is allowed a total of eight ounces of water to be consumed ad libitum during this first 120-180 minutes post-ingestion and is otherwise instructed to follow his normal diet.

Heart rate, systolic blood pressure, and diastolic blood pressure are measured and recorded during at least the first 3 hours post dosing (e.g., at times 30, 60, 90, and 120 minutes post-ingestion, and optionally again at 180 minutes). Heart rate and blood pressure data (including rate pressure product) are analyzed using analysis of variance (ANOVA). Analysis of variance is also performed to assess change from baseline (pre-ingestion) for HR and blood pressure. A correlation analysis (for each test condition independently) on body weight, and the percent change in SBP and DBP at 60 minutes post-ingestion is also performed. Statistical significance is set at p≦0.05.

Cognitive effects: Subjects are monitored from the time right before ingestion until at least twelve hours post-ingestion. Each subject is asked to rate his level of focus, alertness, ability to concentrate, energy level, mood, as well as sense of fatigue, drowsiness, and tiredness, level of twitchiness and restlessness, ability to sit still, and level of anxiety and nervousness. These outcomes can be measured and compared using the visual analog scale. See, e.g., Kaplan et al., Dose-dependent pharmacokinetic and psychomotor effects of caffeine in humans, J. Clin. Pharmacol. 37: 693-703 (1997), which is incorporated herein by reference, and which describes different visual analog scale items (such as energetic/fatigued; thinking slowed down/thinking speeded up; calm/anxious; normal/spacey; relaxed/excited; at ease/nervous) and the statistical analysis employed to quantify these items. Accordingly, subjects are asked to rate parameters on a visual analog scale (e.g., for mood, 0=extremely sad and 10=extremely happy; for energy level, 0=fatigued/no energy and 10=energetic). Each subject is asked to rate these aspects immediately before ingestion, and at time points post ingestion (e.g., at times 30, 60, 90, and 120 minutes, as well as at 3 hours, 4 hours, 6 hours, 8 hours, 12 hours, and 24 hours post-ingestion, and optionally at other time points). For more information on using visual analog scales, see Flint et al., Reproducibility of appetite scores, Int. J. Obesity 24: 38-48 (2000), which is incorporated herein by reference. To quantify effects on mental state, each subject's rating on the visual analog scale is converted to its number value; see Greenblatt et al., Sensitivity to triazolam in the elderly, N. Eng. J. Med. 324: 1691-1698 (1991) and Flint et al. (2000). Mixed or factorial analysis of variance (ANOVA) is performed, with statistical significance set at p≦0.05. Analysis is also performed to measure changes from baseline.

Other tests may be used to further measure cognitive effects, including mental stamina and focus. For example, the digit-symbol substitution test, in which subjects are asked to make as many correct symbol-for-digit substitutions as possible within 2 minutes, is administered twice at the following times: before ingestion, and at 30 minutes, 1 hour, 2 hours, 4 hours, 6 hours, 8 hours, 12 hours, and 24 hours post-ingestion. See, e.g., Greenblatt et al. (1991) and Lapierre et al., The neuropsychiatric effects of aspartame in normal volunteers, J. Clin. Pharmacol. 30: 454-60 (1990), which are incorporated herein by reference. Arithmetic tests can be conducted as an additional measure of cognition, and alertness/focus can be measured with a break-pedal reaction timer to measure reaction time. See Lapierre et al. (1990). The word-list free-recall procedure, which measures the acquisition and recall of information and memory using standardized 16-item word lists, can also be conducted. See Greenblatt et al. (1991) and Lapierre et al. (1990), and further Shader et al., Sedative effects and impaired learning and recall after single oral doses of lorazepam, Clin. Pharmacol. Ther. 39: 526-29 (1986), which is also incorporated herein by reference.

Thermogenesis/energy expenditure: Thermogenesis can be determined pre-dosing and at 30, 60, 90, 120, and 180 minutes post-ingestion, as well as at later time points (e.g., at 3 hours, 4 hours, and 6 hours post-ingestion), and optionally or alternatively at other time points. Thermogenesis can be measured using known methods, such as by using indirect calorimetry. See Johnston et al., Postprandial thermogenesis is increase 100% on a high-protein, low-fat diet versus a high-carbohydrate, low-fat diet in healthy, young women, J. Am. College Nut. 21: 55-61 (2002); Acheson et al., Metabolic effects of caffeine in humans: lipid oxidation or futile cycling?, Am. J. Clin. Nutr. 79: 40-6 (2004), which are incorporated herein by reference. In addition, thermogenesis can be assessed by measuring body temperature (accounting for natural circadian changes in body temperature), as well as by each subject's self-assessment of a thermogenic effect, including by a visual analog scale (e.g., 0=normal/no increase in temperature; 10=very hot). For example, just prior to temperature measurement, each subject may record his sense of feeling cool or hot using VAS. ANOVA analyses are conducted to compare the thermogenic effects of the treatments and the effect of each treatment over time against baseline.

Libido: Each subject assesses his level of libido pre-dosing and following aegeline ingestion at 30 minutes, 60 minutes, 2 hours, 4 hours, 6 hours, 8 hours, 12 hours, and 24 hours post-ingestion. Libido is assessed using a visual analog scale (e.g., 0=low libido; 10=high libido), and these visual analog scale assessments are quantified as described above.

Anabolic effects: The influence of aegeline ingestion on anabolic effects is determined by measuring strength output and dynamic exercise performance. In this study, each subject receives a test composition (either aegeline, at a dose of 200-1000 mg, or placebo) for ingestion. Each subject performs a strength output test, both immediately before ingestion and at 40 minutes post-ingestion. The data are analyzed by a 2 (condition)×2 (time) analysis of variance (ANOVA), with statistical significance set at P≦0.05. Additional subjects may be enrolled to compare the effects of a combination of aegeline and caffeine on strength output.

To measure strength output, each subject first performs a one repetition maximum (1RM) test for the chest press (e.g., using Hammer Strength™ plate-loaded machines, following guidelines from the National Strength and Conditioning Association). This test is performed the day before administration to determine each subject's 1RM. After determination of the 1RMs, the subjects then practice the bench press throw (e.g., using a ProSpot® device; ProSpot Fitness, Norcross, Ga.).

To measure power and local muscular endurance, each subject performs a bench press throw on a device such as a ProSpot® or similar device. The device measures a maximum-effort throw where the bar is stopped at its highest vertical position. Following a warm-up of ˜10% of the subject's predetermined 1RM, each subject performs three maximum throws using 30% of the chest press 1RM, with ninety seconds of rest in between each throw. The best attempt (or highest power) of the three throws is recorded for data analysis. See, e.g., Falvo et al., Efficacy of prior eccentric exercise in attenuating impaired exercise performance after muscle injury in resistance trained men, J. Strength Conditioning Res. 21: 1053-1060 (2007), which is incorporated herein by reference. Kinetic and kinematic data is acquired through the combination of a floor scale (e.g., Roughdeck, Rice Lake Weighing Systems, Rice Lake, Wis.) and a linear velocity transducer (e.g., VP510, Unimeasure, Corvallis, Oreg.) attached to the barbell. Measurements of force and velocity are measured directly by the modified floor scale and linear transducer, respectively. Data are sampled at 1000 Hz and channeled through a 12-bit analog-to-digital converter (e.g., DAS1200Jr; Measurement Computing, Middleboro, Mass.). Data are smoothed using a 4th-order recursive Butterworth digital filter, and power is calculated as the product of force and velocity within the acquisition software (e.g., DataPac 5). After the bench press throws, and to measure upper-body local muscular endurance, each subject performs a maximal number of chest press repetitions to failure with 50% of the previously determined 1RM.

Anabolic effects of aegeline administration can also be measured by assessing each subject's body mass index. See, e.g., Jackson, A. S., and Pollock, M. L., Practical assessment of body composition, Physician and Sportsmedicine, 13: 76-90 (1985), which is incorporated herein by reference. An anabolic effect will result in an increase in the percentage of muscle mass over time.

Each of the methods described above can be performed for test compounds (R)-aegeline and (S)-aegeline, and the effects of caffeine or other substance can be compared to racemic aegeline, (R)-aegeline and (S)-aegeline. Statistical analysis ANOVA is performed to compare the test treatments to each other and to compare the effect of each treatment to baseline.

The invention claimed is:

1. A composition comprising aegeline and one or more substances selected from the group consisting of caffeine, citicoline, bauhinia purpurea L. extract, hemerocallis fulva extract, yohimbe extract, and higenamine.

2. A composition comprising aegeline and citicoline.

3. A composition comprising aegeline, caffeine, bauhinia purpurea L. extract, hemerocallis fulva extract, yohimbe extract, and higenamine.

4. The composition of claim 1, wherein the aegeline is present in an amount ranging from about 15% to about 100% of said composition by weight.

5. The composition of claim 1, wherein the aegeline is present in an amount ranging from about 30 mg to about 3000 mg and the one or more substances is present in an amount ranging from about 0.5 mg to about 500 mg.

6. The composition of claim 1, wherein the aegeline is aegeline succinate.

7. The composition of claim 1, wherein said composition is formulated as an oral dosage form.

8. A method of administering a food or dietary supplement comprising: administering an amount of aegeline orally to a human, wherein the administering step results in one or more effects in the human selected from the group consisting of (a) an increase in mental stamina, (b) an increase in focus, (c) an increase in energy level, (d) an improvement in mood, (e) an increase in thermogenesis, and (f) an increase in muscle output.

9. The method of claim 8, wherein said method further comprises administering one or more substances selected from the group consisting of caffeine, citicoline, bauhinia purpurea L. extract, hemerocallis fulva extract, yohimbe extract, and higenamine.

10. The method of claim 8, wherein the amount of aegeline is an amount ranging from about 30 mg to about 3000 mg.

11. The method of claim 8, wherein the administering step is repeated about daily over a time course ranging from about seven days to about six months.

12. The method of claim 11, wherein said method results in a decreased percentage of fat mass in the human.

13. The method of claim 11, wherein said method results in an increased percentage of muscle mass in the human.

14. The method of claim 8, wherein the food or dietary supplement comprises one or more substances selected from the group consisting of caffeine, citicoline, bauhinia purpurea L. extract, hemerocallis fulva extract, yohimbe extract, and higenamine.

15. The method of claim 14, wherein the administering step is repeated about daily for at least two consecutive days.

16. The method of claim 15, wherein the amount of aegeline is a daily dose ranging from about 30 mg to about 3000 mg.

17. The method of claim 16, wherein the daily dose is gradually increased from an initial daily dose up to a final daily dose.

18. A method of administering a food or dietary supplement comprising: administering an amount of aegeline orally to a human male, wherein the administering step results in an increase in libido in the human male.

19. The method of claim 18, wherein said method further comprises administering one or more substances selected from the group consisting of caffeine, citicoline, bauhinia purpurea L. extract, hemerocallis fulva extract, yohimbe extract, and higenamine.

20. The method of claim 18, wherein the amount of aegeline is an amount ranging from about 150 mg to about 3000 mg.

21. The method of claim 18, wherein the administering step is repeated about daily over a time course ranging from about seven days to about six months.

22. A method for synthesizing aegeline, comprising the steps of
a) preparing an amount of 2-amino-1-(4-methoxyphenyl)ethanone hydrochloride;
b) processing the amount of 2-amino-1-(4-methoxyphenyl)ethanone hydrochloride to form an amount of N-(2-(4-methoxyphenyl)-2-oxoethyl)cinnamamide; and
c) processing the amount of N-(2-(4-methoxyphenyl)-2-oxoethyl)cinnamamide to form an amount of aegeline.

23. The method of claim 22, wherein step (a) comprises reacting an amount of 1-(4-methoxyphenyl)ethanone with N-bromosuccinimide in the presence of heat and in the presence of ethanol.

24. The method of claim 23, wherein step (a) further comprises an amination step comprising adding hexamethylenetetramine.

25. The method of claim 24, wherein the amination step comprises incubating at a temperature of about 25° C. for a time period of about 3 hours after adding hexamethylenetetramine.

26. The method of claim 25, wherein the amination step further comprises adding concentrated hydrogen chloride, followed by stirring at a temperature of about 25° C. for a time period of about 12 hours, followed by reducing reaction volume and cooling to a temperature of about 5° C.

27. The method of claim 22, wherein step (b) comprises reacting the amount of 2-amino-1-(4-methoxyphenyl)ethanone hydrochloride with cinnamoyl chloride in the presence of triethylamine and dichloromethane.

28. The method of claim 27, wherein the reaction step in step (b) comprises stirring at a temperature of about 25° C. for time period of about 3 hours.

29. The method of claim 22, wherein step (c) comprises reacting the amount of N-(2-(4-methoxyphenyl)-2-oxoethyl)cinnamamide with sodium borohydride in the presence of ethanol.

30. The method of claim 29, wherein the reaction step in step (c) is performed at temperature of about 10° C. for a time period of about 2 hours and is followed by concentrating volume, adding water, and stirring at a temperature of about 5° C. to form a precipitated solid.

31. The method of claim 30, wherein the precipitated solid is boiled in alcohol and then cooled to a temperature of about 5° C. to form the amount of aegeline.
